(19) United States
(12) Patent Application Publication
(10) Pub. No.: US 2024/0174623 A1
(43) Pub. Date: May 30, 2024

(54) Build UP Film Curable Compositions

(71) Applicant: AGC Multi Material America, Inc., Tempe, AZ (US)

(72) Inventors: Preeya Kuray, Tempe, AZ (US); Caleb Ancharski, Tempe, AZ (US); Yoji Nakajima, Tempe, AZ (US); Mark Derwin, Tempe, AZ (US); Thomas Fitzgerald McCarthy, III, Tempe, AZ (US); Takefumi Abe, Tempe, AZ (US)

(21) Appl. No.: 18/508,981

(22) Filed: Nov. 14, 2023

Related U.S. Application Data
(60) Provisional application No. 63/425,780, filed on Nov. 16, 2022.

(51) Int. Cl. C07D 265/14; C07D 239/04; C08F 4/04; C08F 4/34; C08K 7/22

(52) U.S. Cl. CPC C07D 265/14; C07D 239/04; C08F 4/04; C08F 4/34; C08K 7/22; C08K 2201/003

(57) ABSTRACT

This disclosure relates to a curable composition that includes at least one maleimide-containing compound or benzoxazine compound, at least one low dielectric loss polymer or a hydrogenated derivative thereof, at least one filler, and at least one radical initiator. This disclosure also relates to using the composition to form a film, a laminate, and/or a circuit board.

The present application claims the benefit of U.S. Provisional Application No. 63/425,780, filed Nov. 16, 2022, which is incorporated herein by reference in its entirety.

### TECHNICAL FIELD

The present disclosure relates to curable compositions, as well as related methods, films, laminates, and circuit boards.

### BACKGROUND

A printed circuit board (PCB) can include build up layers of circuitry made from dielectric films. The dielectric films can include polymeric materials with or without inorganic particulates. The polymeric materials can be thermoplastic or thermosetting. Polytetrafluoroethylene (PTFE) and ceramic filled PTFE are examples of thermoplastic polymers that can be used to create build-up dielectric layers. However, PTFE-based materials face challenges such as low dimensional stability, high coefficients of thermal expansion, high lamination temperatures, high costs, and low yields.

Epoxy materials can be used to prepare thermosetting polymers to form build-up dielectric layers. Highly dense circuits formed by laser ablation of thin dielectric layers is commonly done by using ceramic filled epoxy materials. Ceramic particulates are well known in the art to reduce the coefficient of thermal expansion of free standing films. However, conventional build-up dielectric layers generally suffer from high electrical loss. For example, build-up dielectric layers formed by using epoxy materials can have relatively high loss tangent (Df) near 0.02. Build-up dielectric layers with lower electrical loss can be made by using cyanate ester/epoxy blends to reach a loss tangent closer to 0.007. In addition, build-up dielectric layers having a loss tangent from 0.004 to 0.006 can be achieved by using extremely high levels of ceramic fillers and flame retardants. However, these build-up dielectric layers tend to be very stiff and have high tensile modulus.

Printed circuits boards have always suffered from the complex integration of different polymeric materials, various metallization layers, the inconsistency of the distribution of metallic layers, and the interconnection of the various layers in the z axis with a certain geometry and the inconsistency in the X/Y planes. Each of the various materials in a PCB has its own coefficients of thermal expansion, tensile strength, flexural strength, and compression strengths/modulus. In addition, printed circuits are typically reflowed at 260° C. Many of these engineering properties are not constant from the room temperature to the above reflow temperature. Polymeric materials, for example, can have very different mechanical properties with an increasing temperature. The result is that a complex amount of localized stress at a given temperature can build up in the circuit board and lead to failure at elevated temperatures. Typical failures include delamination and cracks.

### SUMMARY

The present disclosure is based on the unexpected discovery that certain curable compositions that include either a maleimide-containing compound or a benzoxazine compound, or the combination thereof, and a low dielectric loss polymer can form a dielectric layer or film (e.g., a build-up film having multiple layers) having superior electrical properties (e.g., low Dk and Df), improved flame resistance (e.g., achieving UL94 V-0 flammability performance), improved mechanical properties (e.g., low tensile modulus), compared to a conventional dielectric film. For example, a dielectric film made from a curable composition described herein can have a relatively low Dk/Df (which results in a relatively low dielectric loss) and a relatively low tensile modulus (such that the film does not easily for cracks). In addition, such a dielectric film can be made from a curable composition described herein at a relatively low cost and a relatively high yield for high volume commercial manufacturing.

In one aspect, the present disclosure features curable compositions that include (1) at least either one maleimide-containing compound comprising a bismaleimide compound or a polymaleimide, or a benzoxazine compound, or a combination thereof; (2) at least one low dielectric loss polymer or a hydrogenated derivative thereof, the at least one low dielectric loss polymer including a poly(phenylene ether), or a copolymer comprising a styrene monomer unit, an ethylene monomer unit, a propylene monomer unit, a butylene monomer unit, a butadiene monomer unit, an isoprene monomer unit, a divinylbenzene monomer unit, a pyrimidine monomer unit, or a pyridazine monomer unit; (3) at least one filler; and (4) at least one radical initiator.

In another aspect, the present disclosure features curable compositions that include at least one nitrogen containing compound such as a maleimide, a bismaleimide compound or a polymaleimide, or a benzoxazine, or a combination thereof, and at least one filler, at least one radical initiator, and an organic solvent.

In another aspect, the present disclosure features a film (e.g., a free-standing or supported film) prepared from a curable composition described herein.

In another aspect, the present disclosure features an article that includes a carrier and at least one layer supported by the carrier, in which the least one layer includes a film prepared from a curable composition described herein.

In another aspect, the present disclosure features an article that includes a fiberglass based substrate core, a glass core with or without metallized through vias, or a TSV; and from 1 to 10 build up film layers that are laminated on top, bottom or both sides of the substrate core, glass core, or TSV, together with copper metallization such that circuitry are created that is flame retardant and passes UL VO.

In another aspect, the present disclosure features a laminate that includes first and second films or a plurality of films; and a woven or non-woven substrate between the first and second films; in which at least one of the first and second films is a film prepared from a curable composition described herein.

In another aspect, the present disclosure features a circuit board (e.g., a printed circuit board) for use in an electronic product that includes a laminate described herein.

The details of one or more embodiments of the disclosed compositions and methods are set forth in the description below. Other features, objects, and advantages of the disclosed compositions and methods will be apparent from the description and the claims.

### DETAILED DESCRIPTION

In general, the present disclosure is directed to curable compositions that include at least one maleimide-containing compound or benzoxazine compound, or combination thereof, at least one low dielectric loss polymer or a hydrogenated derivative thereof, at least one filler, and at least one radical initiator. In some embodiments, the curable compositions described herein can be substantially free of a halogen (e.g., F, Cl, Br, or I). In some embodiments, the polymer described herein can be a homopolymer or a copolymer (e.g., a random copolymer, a graft copolymer, an alternating copolymer, or a block copolymer). In some embodiments, the block copolymer described herein can be a diblock copolymer, a triblock, or a tetrablock copolymer,

In some embodiments, the curable compositions described herein can include at least one (e.g., two or three or more) maleimide-containing compound. In some embodiments, the maleimide-containing compound can include aliphatic moieties, aromatic moieties, and a combination thereof. In some embodiments, the maleimide-containing compound can include at least one C₄-C₄₀ alkyl group, at least one C₄-C₄₀ alkylene group, at least one aryl group, or at least one heteroaryl group. In some embodiments, the maleimide-containing compound can have a long aliphatic chain (e.g., at least one C₄-C₄₀ alkyl group or at least one C₄-C₄₀ alkylene group). In some embodiments, the maleimide-containing compound can include a total of 10-200 carbon atoms. In some embodiments, the maleimide-containing compound can be linear, branched, cyclic, or a combination thereof. Without wishing to be bound by theory, it is believed that a dielectric film made from such a maleimide-containing compound can have a relatively low modulus.

In some embodiments, the maleimide-containing compound can be a monomer (e.g., a bismaleimide or a monomaleimide such as citraconimide) or a polymer (e.g., a polymaleimide or a bismaleimide polymer). Examples of suitable maleimide-containing monomers include 1,6-bis(maleimido)hexane, 1,10-bis(maleimide)decane, 1,3-phenylene bismaleimide, 3,3′-dimethyl-5,5′-diethyl-4,4′-diphenylmethane bismaleimide, m-xylenebismaleimide, N,N′-bismaleimido-4,4′-diphenylmethane, 1,6-bismaleimido(2,2,4-trimethyl)hexane, 4-methyl-1,3-phenylene bismaleimide, 1,3-bis(3-maleimidophenoxy)benzene, 1,3-bis(4-maleimidophenoxy)benzene, 1,3-bis(citraconimidomethyl)benzene, bisphenol A diphenylether bismaleimide, or 2,2′-bis-[4-(4-maleimidophenoxy)phenyl]propane. A further example is DIC NE-X-9470S which is an alkylmodified bismaleimide.

Examples of suitable maleimide-containing polymers include polymaleimides (e.g., poly(phenylmethanmaleimide)) and bismaleimide polymers (e.g., bismaleimide-terminated polymers). An example of the maleimide-containing polymer described herein can be a polymaleimide of formula (1):

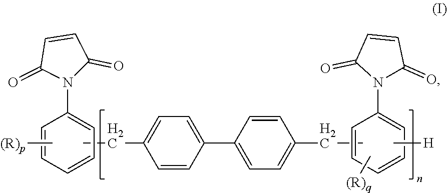

in which each R, independently, is hydrogen, 1-12 alkyl e.g., methyl), or phenyl; n is an integer from 1 to 100; p is an integer from 0 to 4; and q is an integer from 0 to 3. Examples of the bismaleimide-terminated polymer described herein can include 1,2-bis(octylmaleimide)-3-octyl-4-hexyl)cyclohexyl oligomers, imide-extended bismaleimide oligomers, and maleimide terminated 2-[8-(3-hexyl-2,6-dioctylcyclohexyl)octyl]pyromellitic diimide oligomers. In some embodiments, the maleimide-containing polymers described herein can include 2-10 monomer repeating units.

Examples of suitable maleimide-containing monomers or polymers include compounds of formulas (1), (2), and (3):

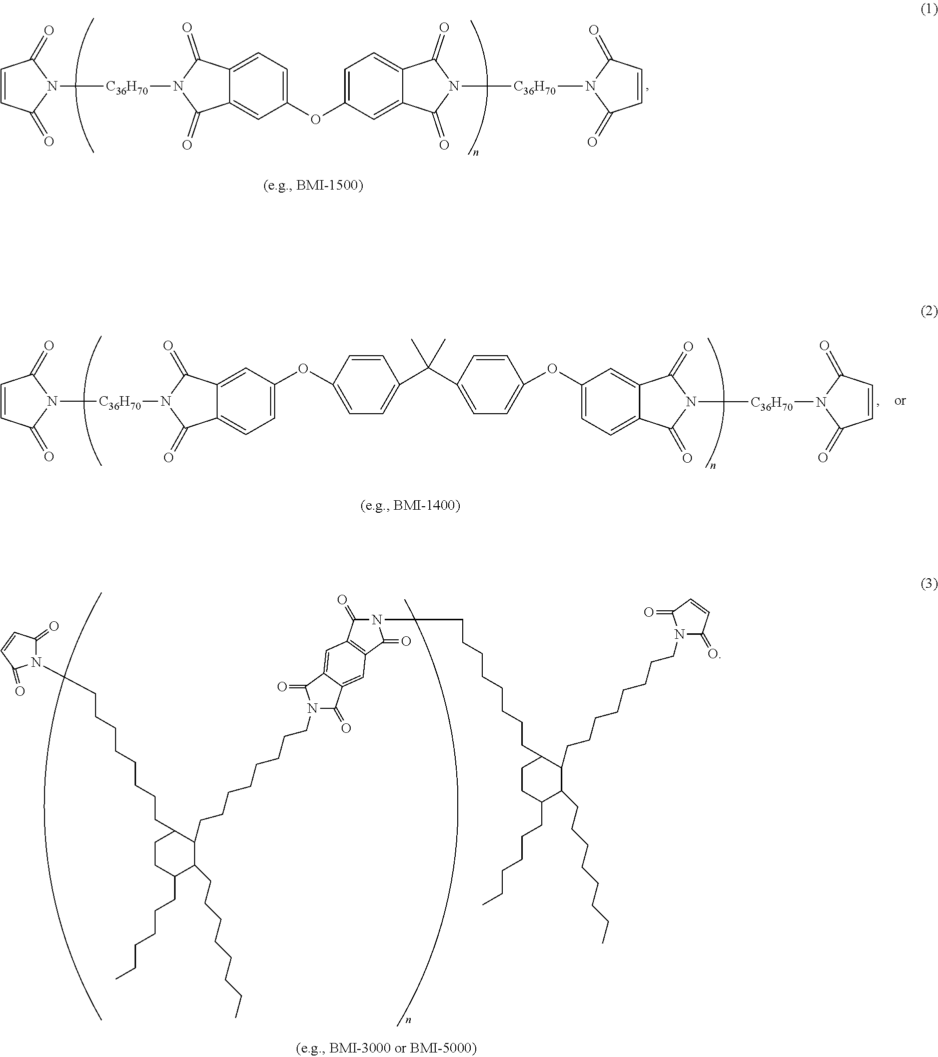

In some embodiments, n is an integer from 1 to 10. In some embodiments, n is an integer with an average value of about 1.3. In some embodiments, n is an integer with an average value of about 3.1. Other bismaleimide compounds include BMI-689, BMI-1550, BMI-2500, BMI-2560, BMI-6000, and BMI-6100 available from Designer Molecules Inc.

In some embodiments, the maleimide-containing compound described herein can include those modified with another agent, such as triallylisocyanurate, tricyanurate, benzoxazines, or cyanate ester resins. Examples of suitable cyanate ester resins include 1,1′-bis(4-cyanatophenyl)ethane, bis(4-cyanato-3,5-dimethylphenyl)methane, 1,3-bis(4-cyanatophenyl-1 (1-methylethylidene)benzene, the cyanate ester adducts of phenol and dicyclopentadiene, and the cyanate esters of phenol-formaldehyde oligomers.

In another embodiment, the benzoxazine can be used with the absence of a bismaleimide or in combination. The benzoxazines described herein can include monofunctional, bifunctional, trifunctional, and polyfunctional benzoxazines. Examples of suitable benzoxazines include those containing one or more cyclopentadiene, bisphenol F, bisphenol A, phenophthalein, or thiodiphenol group. A further example of a benzoxazine is P-d Benzoxazine available from Shikoku Kasei described by the following representation:

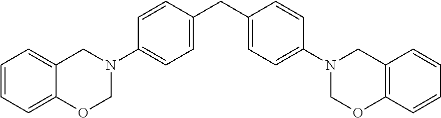 [SMILES: c1ccc2c(c1)CN(c1ccc(Cc3ccc(N4COc5ccccc5C4)cc3)cc1)CO2]

A preference is given to non-aniline benzoxazines although this is not a requirement. A further example is a diallyl-substituted benzoxazine offered by Kolon Industries (KZH-5031). The X group can be an alkylene group, an arylene group with any kind of substitution around the arylene ring, or a combination of aromatic and aliphatic groups. Examples of X include, but are not limited to, phenylene, naphthylene, biphenylene, isopropylidene, methylene, or linear alkylenes from 1-20 carbon atoms. X could also be a heteroatom such as oxygen.

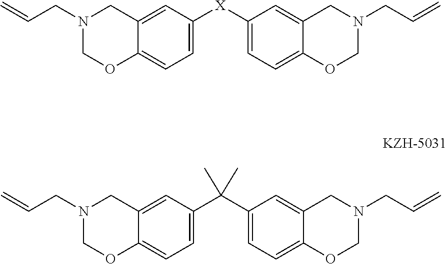

In some embodiments, the benzoxazine compound is a compound of the following structure:

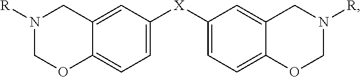

wherein X is an alkylene group, an optionally substituted arylene group, a heteroatom, or a combination of aromatic and aliphatic groups; and R is optionally substituted alkyl, optionally substituted cycloalkyl, allyl, optionally substituted C₆₋₂₂ aryl, optionally substituted 5-22 membered heteroaryl, or an oligomer. In some embodiments, X is phenylene, naphthylene, biphenylene, isopropylidene, methylene, a linear C₂₋₂₀ alkylene group, or a heteroatom. In some embodiments, X is isopropylidene or methylene. In some embodiments, X is oxygen. In some embodiments, R is allyl, phenyl, naphthyl, biphenyl, benzyl, or alkyl-substituted phenyl.

In some embodiments, the benzoxazine compound is a compound of one of the following structures:

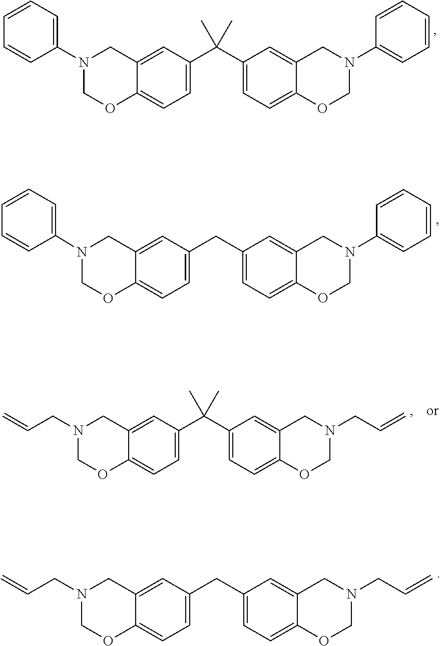

A further embodiment is to employ monofunctional benzoxazines that can be used for flow control. Reactive diluents must be carefully chosen because the improper choice can lead to premature curing, a reducing curing window, phase separation in the prepreg state, outgassing, higher CTE values, etc.

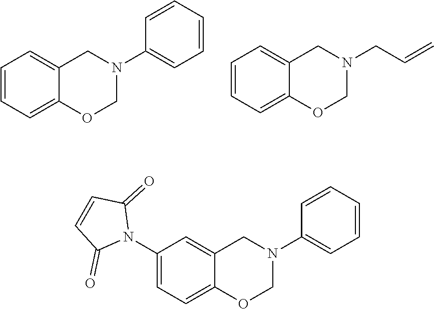

Examples of monobenzoxazines are depicted above.

In some embodiments the benzoxazine is employed in the absence or in combination with the bismaleimide. Benzoxazines and bismaleimides are nitrogen containing compounds that offer the benefit of natural flame retardancy. Fully aromatic bismaleimides or benzoxazines will offer the highest levels of flame retardancy comparted to partially aromatic or fully aliphatic benzoxazines or bismaleimides.

In some embodiments, the maleimide-containing compound described herein is present in an amount of from at least about 1 wt % (e.g., at least about 2 wt %, at least about 4 wt %, at least about 5 wt %, at least about 6 wt %, at least about 8 wt %, at least about 10 wt %, at least about 12 wt %, at least about 14 wt %, at least about 16 wt %, at least about 18 wt %, at least about 20 wt %, at least about 25 wt %, at least about 30 wt %, at least about 35 wt %, or at least about 40 wt %) to at most about 50 wt % (e.g., at most about 45 wt %, at most about 40 wt %, at most about 35 wt %, at most about 30 wt %, at most about 25 wt %, at most about 20 wt %, at most about 18 wt %, at most about 16 wt %, at most about 15 wt %, at most about 14 wt %, at most about 12 wt %, or at most about 10 wt %) of the solid content of the curable compositions described herein.

In some embodiments, the benzoxazine containing compound described herein is present in an amount of from at least about 1 wt % (e.g., at least about 2 wt %, at least about 4 wt %, at least about 5 wt %, at least about 6 wt %, at least about 8 wt %, at least about 10 wt %, at least about 12 wt %, at least about 14 wt %, at least about 16 wt %, at least about 18 wt %, at least about 20 wt %, at least about 25 wt %, at least about 30 wt %, at least about 35 wt %, or at least about 40 wt %) to at most about 50 wt % (e.g., at most about 45 wt %, at most about 40 wt %, at most about 35 wt %, at most about 30 wt %, at most about 25 wt %, at most about 20 wt %, at most about 18 wt %, at most about 16 wt %, at most about 15 wt %, at most about 14 wt %, at most about 12 wt %, or at most about 10 wt %) of the solid content of the curable compositions described herein. As used herein, the term “solid content” refers to the total content of a curable composition after the solvent for the curable composition is removed.

In some embodiments, the fully cured build up film compound described herein has a relatively low tensile modulus. For example, fully cured build up film can have a tensile modulus of from at most about 20 GPa (e.g., at most about 10 GPa, at most about 1 GPa [all measurements at room temperature], or at most about 0.5 GPa to a least about 0.01 GPa (e.g., at least about 0.05 GPa, at least about 0.10 GPa). When measured at 260 C, the reflow temperature of lead free solder, it is preferred that the tensile modulus be from 0.01 to 0.5 GPa, more preferred be from 0.02 to 0.25 GPa, most preferred that the tensile modulus be 0.025 to 0.150 GPa. Without wishing to be bound by theory, it is believed that the curable composition containing a low modulus compound or resin can result in a film that has a reduced internal stress (e.g., after repeated heating and cooling cycles) and has improved resistance to failure (e.g., delamination or forming cracks) and has a reduced chance to contribute to the failure of other parts of a high density printed circuit board. In semiconductor packaging warpage is a large concern relative to large packages. Low modulus packaging materials lead to less internal stress and are less prone to crack formation or warpage.

In some embodiments, the curable compositions described herein can optionally include at least one (e.g., two or three or more) low dielectric loss polymer or a hydrogenated derivative thereof. The hydrogenated derivative can be a partially hydrogenated low dielectric loss polymer or a fully hydrogenated low dielectric loss polymer (i.e., without any residue olefin group).

In some embodiments, the low dielectric loss polymer described herein is curable or cross-linkable (e.g., either in the presence of an initiator or heat). In some embodiments, the low dielectric loss polymer can include at least two carbon-carbon double bonds, which can be either at a polymer chain end (i.e., in an end group) or in the middle of a polymer chain (e.g., in a side chain). As used herein, the “carbon-carbon double bond” refers to a non-aromatic carbon-carbon double bond, such as an ethylenic group or a vinyl group. For example, the low dielectric loss polymer can be terminated with a functional group containing a maleimide, styrene, allyl, allyl ether, acrylate, methacrylate, isocyanurate, or benzoxazine group (which includes a carbon-carbon double bond).

In some embodiments, the low dielectric loss polymer described herein does not include a carbon-carbon double bond, but includes a functional group that can generate radicals under heat or can react through chain transfer, which can be crosslinked. Without being bound by theory, an example of such a functional group is a substituted or unsubstituted aromatic group. In some embodiments, a thermosetting resin which can be used may not include any crosslinkable vinyl, non-aromatic carbon-carbon double bond. An example of such a thermosetting resin is a polymer (e.g., a copolymer) including a methylstyrene monomer unit, such as poly(methylstyrene) which can crosslink through chain transfer reactions.

In some embodiments, the low dielectric loss polymer described herein can be a poly(arylene ether) polymer. In some embodiments, the poly(arylene ether) polymer described herein can include at least one (e.g., two or three or more) first monomer unit and optionally at least one (e.g., two or three or more) second monomer unit different from the first monomer unit. The phrase “monomer unit” mentioned herein refers to a group formed from a monomer and is used interchangeably with “monomer repeat unit” known in the art. In some embodiments, the copolymer includes the first monomer unit and the optional second monomer unit only and does not include any other monomer unit.

In some embodiments, the poly(arylene ether) polymer described herein can include a nitrogen atom in its polymer structure. In some embodiments, the poly(arylene ether) polymer described herein can include a monomer unit containing a pyridazine, pyrimidine, or pyrazine group. Examples of such a polymer can include a monomer unit having a chemical structure shown in one of the formulas (II)-(IV) below:

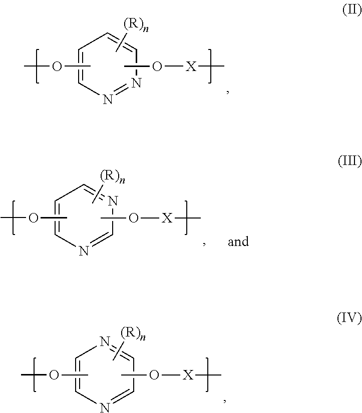

in which n is 0, 1, or 2; each R independently is a monovalent hydrocarbon group having 1 to 20 carbon atoms (e.g., C₁-C₂₀ alkyl or C₆-C₂₀ aryl), CN, NO₂, or N(R′R″), where each of R′ and R″ is C₁-C₂₀ alkyl or aryl; and X is a divalent organic group containing at least one (e.g., two or three) aromatic group (e.g., a phenylene group). In some embodiments, X in formulas (II)-(IV) can have a chemical structure shown in formula V below:

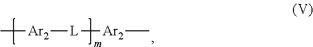

in which m is 0, 1, 2, 3, 4, or 5; each of Ar and Ar₂ independently is an aromatic group (e.g., a phenylene group) optionally substituted by one or more (e.g., 1, 2, 3, or 4) C₁-C₂₀ alkyl; and L is a single bond, —O—, —S—, —N(Ra)—, —C(O)—, —SO₂—, —P(O)—, or a divalent hydrocarbon group having 1 to 20 carbon atoms (e.g., C₁-C₂₀ alkylene or

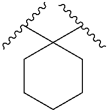 [SMILES: *C1(*)CCCCC1]

optionally substituted by one or more (e.g., 1, 2, 3, 4, or 5) C₁-C₁₀ alkyl).

In some embodiments, the poly(arylene ether) polymer described herein can include a monomer unit having a chemical structure shown in one of the formulas (4)-(8) below:

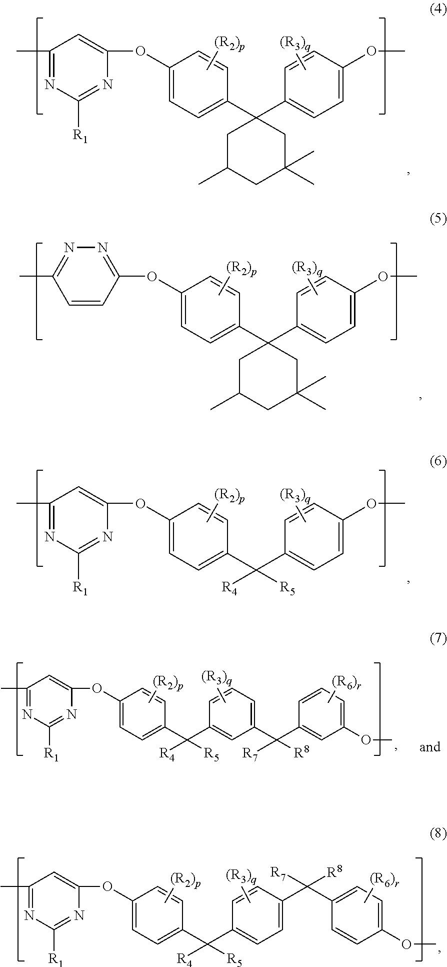

in which R₁ is H, C₁-C₁₀ alkyl, or aryl; each of R₂, R₃, and R₆, independently, is C₁-C₁₀ alkyl; each of R₄, R₅, R₇, and R₈ is H or C₁-C₁₀ alkyl; p is an integer from 0 to 4; q is an integer from 0 to 4; and r is an integer from 0 to 4.

Examples of the poly(arylene ether) polymer described herein can include a monomer unit having one of the following chemical structures:

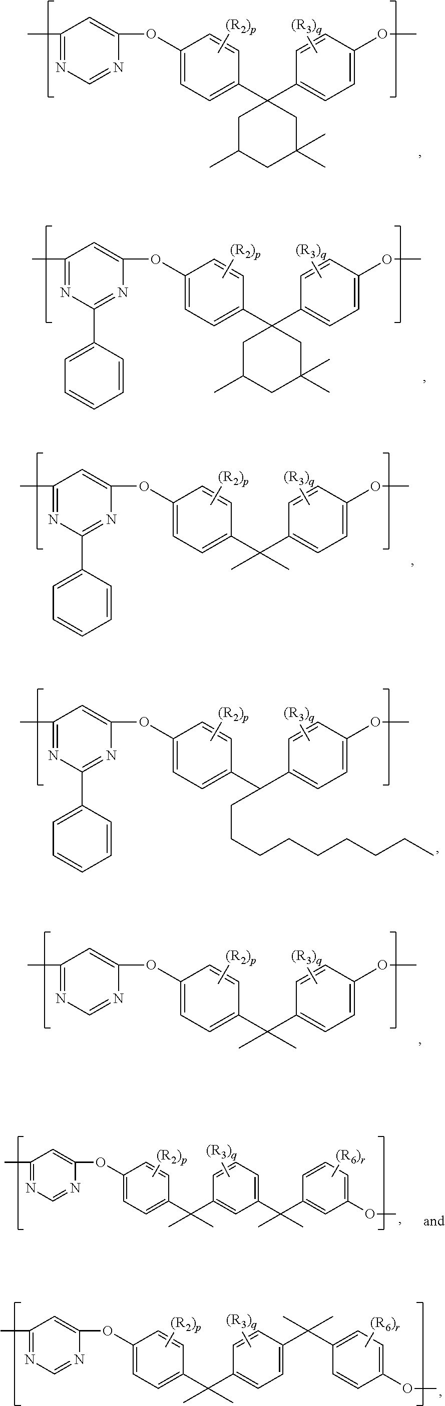

in which p, q, r, R₂, R₃, and R₆ are defined above. A specific example of such a polymer has the following chemical structure:

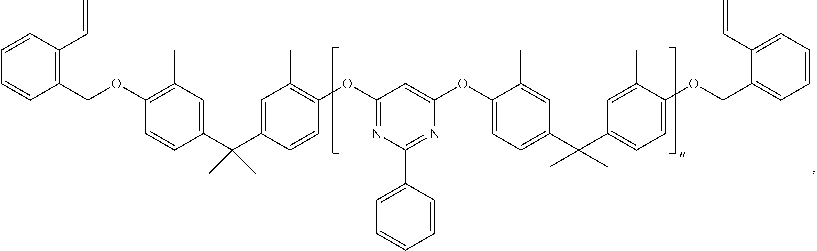

in which n is an integer from 1 to 100. Commercially examples of the poly(arylene ether) polymer described herein include functionalized poly(pyrimidine aryl ether) copolymers JSR HC-21 and HC-30 available from Japanese Synthetic Rubber Corporation.

In some embodiments, the low dielectric loss polymer described herein can be a poly(phenylene ether). In some embodiments, the poly(phenylene ether) is of formula (VI):

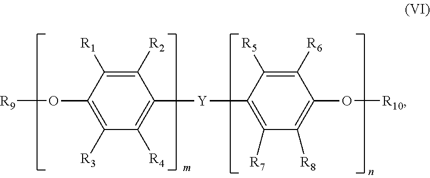

in which each of m and n, independently, is an integer from 1 to 100; each of R₁, R₂, R₃, R₄, R₅, R₆, R₇, and R₈, independently, is hydrogen or C₁-C₁₂ alkyl (e.g., methyl); each of R₉ and R₁₀, independently, is an end group containing a carbon-carbon double bond; and Y is a single bond, —C(O)—, —C(S)—, —S(O)—, —S(O)₂—, —C(R₁R₂)—, or

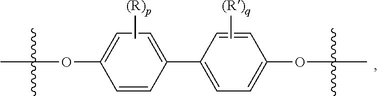

in which each of R and R′, independently, is hydrogen or C₁-C₁₂ alkyl (e.g., methyl), p is an integer from 0 to 4, and q is an integer from 0 to 4. In some embodiments, the poly(phenylene ether) can have one of the following groups in one or both of the end groups:

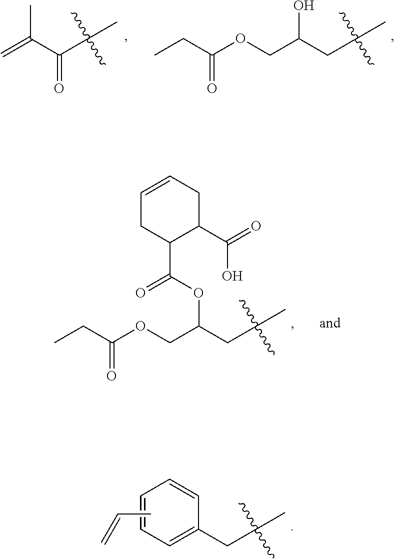

Examples of suitable poly(phenylene ether)s of formula (VI) include:

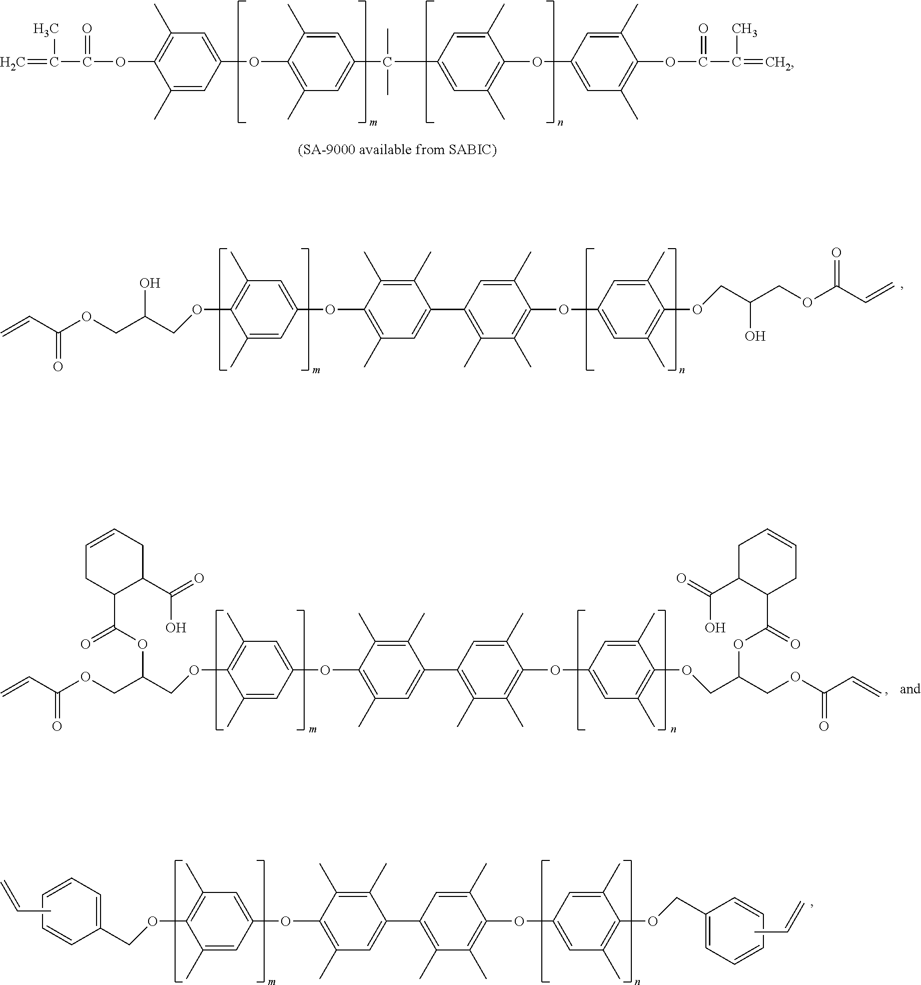

in which m is an integer from 1-100 and n is an integer from 1-100. Other examples of suitable poly(phenylene ether)s include those modified with butadiene, isoprene, styrene, alpha-methylstyrene, and their partially or fully hydrogenated derivatives.

In some embodiments, the low dielectric loss polymer described herein can be an elastomer (e.g., a non-polar elastomer). In some embodiments, the elastomer described herein can include an ethylene monomer unit, a propylene monomer unit, a butylene monomer unit, an isobutylene monomer unit, a butadiene monomer unit, an isoprene monomer unit, a cyclohexene monomer unit, a substituted or unsubstituted styrene (e.g., an alkyl substituted styrene such as methylstyrene, or a divinylbenzene) monomer unit, or a combination thereof. Examples of suitable elastomers include a polyethylene, a polypropylene, a polybutadiene, a polystyrene, a poly(styrene-co-butadiene) (SB) copolymer, a polydivinylbenzene copolymer, a poly(styrene-ethylene-butylene-styrene) (SEBS) copolymer, a poly(styrene-ethylene-propylene-styrene) (SEPS) copolymer, a poly(styrene-ethylene-(ethylene-propylene)-styrene) (SEEPS) copolymer, a poly(styrene-butadiene-styrene) (SBS) copolymer, a poly(butadiene-styrene-butadiene) (BSB) copolymer, a poly(styrene-isoprene-styrene) (SIS) copolymer, a poly(styrene-propylene-styrene) (SPS) copolymer, and a poly(ethylene-propylene-diene) (EPD) copolymer. In some embodiments, the styrene monomer unit of the elastomers described above can be substituted by one or more alkyl (e.g., methyl), aryl (e.g., phenyl), vinyl, allyl, vinyl functionalized silyl, bismaleimide, acrylate, methacrylate, alpha-methyl, and maleic anhydride groups. In some embodiments, the elastomers described herein can be partially or fully hydrogenated. Modified polymers of polydivinylbenzene such as copolymers thereof with styrene or triblock polymers comprising divinylbenzene, styrene, and ethylene are also suitable curable polymers that can be used that offer low electrical loss and some elastomeric properties. A further example is a modified polydivinylbenzene LME11613 offered by Huntsman that can be described by the following chemical representations:

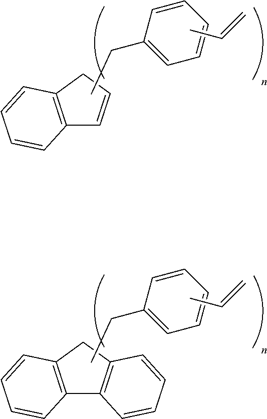

Without wishing to be bound by theory, it is believed that the proper choice of the elastomer can improve the thermal properties (e.g., oxidation resistance), mechanical properties (e.g., copper peel strength and/or inner layer bond strength), or electrical properties (e.g., Dk and/or Df) of the curable compositions described herein. The proper choice of elastomer should allow a targeted coefficient of thermal expansion to be achieved based on the total composition of the dielectric film. In addition, without wishing to be bound by theory, it is believed that the elastomeric component contributes to ductility, is compliant, and can act as a stress relief material in a polymeric build-up film. Further, without wishing to be bound by theory, it is believed that the elastomeric component improves handleability, acts as a toughener, and helps a dielectric film resist any crack initiation and propagation. For example, when a build-up, multilayer, polymeric film is applied adjacent to another material of much higher stiffness or another material having a much higher or lower CTE, the elastomeric component can help mitigate stress prevent any crack formation, mitigate any stresses causing warpage.

In some embodiments, the low dielectric loss polymer described herein (e.g., a poly(arylene ether) polymer or an elastomer) can have a Dk ranging from at most about 4.0 (e.g., at most about 3.8, at most about 3.6, at most about 3.5, at most about 3.4, at most about 3.2, at most about 3, at most about 2.8, at most about 2.6, at most about 2.5, at most about 2.4, at most about 2.2, at most about 2, at most about 1.8, or at most about 1.6) to at least about 1 (e.g., at least about 1.5 or at least about 2) at 10 GHz.

In some embodiments, the low dielectric loss polymer described herein (e.g., a poly(arylene ether) polymer or an elastomer) can have a dielectric loss or dissipation factor (Df) of from at most about 0.005 (e.g., at most about 0.0045, at most about 0.004, at most about 0.0035, at most about 0.003, at most about 0.0028, at most about 0.0026, at most about 0.0025, at most about 0.0024, at most about 0.0022, at most about 0.002, at most about 0.0018, at most about 0.0016, at most about 0.0015, at most about 0.0014, at most about 0.0012, at most about 0.001, or at most about 0.0008) to at least about 0.0005 (e.g., at least about 0.0006 or at least about 0.0008) at 10 GHz.

In some embodiments, the low dielectric loss polymer is present in an amount of from at least about 1 wt % (e.g., at least about 2 wt %, at least about 3 wt %, at least about 4 wt %, at least about 5 wt %, at least about 6 wt %, at least about 7 wt %, at least about 8 wt %, at least about 9 wt %, at least about 10 wt %, at least about 12 wt %, at least about 14 wt %, at least about 15 wt %, at least about 16 wt %, at least about 18 wt %, at least about 20 wt %, or at least about 25 wt %) to at most about 30 wt % (e.g., at most about 25 wt %, at most about 20 wt %, at most about 18 wt %, at most about 16 wt %, at most about 15 wt %, at most about 14 wt %, at most about 12 wt %, or at most about 10 wt %) of the solid content of the curable compositions described herein. Without wishing to be bound by theory, it is believed that a curable composition containing the low dielectric loss polymer described herein can have superior electrical properties (e.g., low Dk or Df) and flame resistance.

In some embodiments, the curable compositions described herein can include at least one (e.g., two or three or more) filler (e.g., inorganic filler). In general, the filler is not soluble in an organic solvent. Examples of suitable fillers include silica, alumina, quartz, titanium oxide, boron nitride, barium titanate, barium strontium titanate, and polymer particles. In some embodiments, the polymer particles can include a fluoro-containing polymer (e.g., poly(tetrafluoroethylene), crosslinked PTFE, poly(divinylbenzene), poly(divinylbenzene-styrene), poly(divinylbenzene-styrene-ethylene), polyimide, polyetherimide, polyetheretherketone, polybenzimidazole, and polydicyclopentadiene. In some embodiments, the particles can include cross-linked polymers. In some embodiments, the filler can be in the form of beads, particles, or flat platelets, or powders. Without wishing to be bound by theories, it is believed that the filler can improve the mechanical properties, thermal conductivity, dimensional stability, and electrical properties, and/or can lower the coefficient thermal expansion (CTE) and costs of the curable compositions described herein.

In some embodiments, the filler described herein can be surface treated. For example, the filler can be treated by a surface treatment agent that includes one or more of the following groups: silyl, polymethylsilsesquioxane, vinyl, epoxy, n-alkyl with up to 20 carbons (such as an octyl group, dodecyl group, or propyl group, i.e., tripropyl), phenyl, amino, acrylic, methacrylic, isocyanate, phenyl, and fluorine.

In some embodiments, the filler described herein can include hollow silica particles (e.g., hollow silica beads) or hollow polymer particles. Without wishing to be bound by theories, it is believed that using hollow particles as a filler can improve the electrical properties (e.g., reducing the Dk and/or Df) of the curable compositions described herein compared to using solid particles as a filler. In some embodiments, the curable compositions described herein can include one or more (e.g., two or three) non-hollow, solid fillers and/or one or more (e.g., two or three) hollow fillers. In some embodiments, the curable compositions described herein do not include a hollow filler. It is preferred that the hollow filler have a strong enough shell that it can tolerate some mechanical stress without breakage.

In some embodiments, the filler (e.g., hollow silica particles) described herein can have a relatively high purity. For example, a silica filler (e.g., hollow silica particles) can include at least about 95 wt % (e.g., at least about 96 wt %, at least about 97 wt %, at least about 98 wt %, at least about 99 wt %, or at least about 99.5 wt %) silicon dioxide. Without wishing to be bound by theory, it is believed that using a high purity hollow filler can lower the Df of a film made from such a filler and can result in less metal contamination that may cause reliability issues.

In some embodiments, the filler (e.g., hollow silica particles) described herein can have a relatively small density. For example, the filler (e.g., hollow silica particles) can have a density of at most about 1.5 g/cm³ (e.g., at most about 1.4 g/cm³, at most about 1.2 g/cm³, at most about 1 g/cm³, at most about 0.8 g/cm³, at most about 0.6 g/cm³, at most about 0.5 g/cm³, or at most about 0.4 g/cm³). Without wishing to be bound by theory, it is believed that a low density filler can lower the Df and/or Dk of a film made from such a filler.

In some embodiments, the filler described herein can include solid, non-porous particles (e.g., solid silica particles), which are not prone to water or other chemical uptake. In some embodiments, a process of printed circuit board fabrication involves process steps during which a filler should not be prone to uptake by solvents, metal particulates, organic chemicals, resins, oligomers or polymers, surfactants, other inorganic chemicals such as glass etchants. In some embodiments, solid fillers can have a density ranging from about 1 g/cm³ to about 4 g/cm³.

In some embodiments, the filler described herein can have a relatively low dielectric constant (Dk) and/or a relatively low dissipation factor (Df). For example, the filler described herein can have a Dk ranging from at most about 4.0 (e.g., at most about 3.8, at most about 3.6, at most about 3.5, at most about 3.4, at most about 3.2, at most about 3, at most about 2.8, at most about 2.7, at most about 2.6, at most about 2.5, at most about 2.4, at most about 2.2, at most about 2, at most about 1.8, or at most about 1.6) to at least about 1 (e.g., at least about 1.5) at 10 GHz. In some embodiments, the filler described herein can have a dissipation factor (Df) ranging from at most about 0.002 (e.g., at most about 0.0018, at most about 0.0016, at most about 0.0015, at most about 0.0014, at most about 0.0012, at most about 0.001, or at most about 0.0008) to at least about 0.0005 (e.g., at least about 0.0006, at least about 0.0008, or at least about 0.001) at 10 GHz.

In some embodiments, the filler mentioned herein can have a relatively small particle size. For example, the filler can have a particle size D50 value of at most about 10 μm (e.g., at most about 8 μm, at most about 6 μm, at most about 5 μm, at most about 4 μm, or at most about 2 μm) and/or at least about 0.5 μm (e.g., at least about 0.8 μm, at least about 1 μm, at least about 1.5 μm, or at least about 2 μm). Without wishing to be bound by theory, it is believed that using a filler having a relatively small size can reduce the defects in a film made from such a filler during a PCB process.

In some embodiments, the at least one filler is present in an amount of from at least about 40 wt % (e.g., at least about 45 wt %, at least about 50 wt %, at least about 55 wt %, at least about 60 wt %, at least about 65 wt %, or at least about 70 wt %) to at most about 85 wt % (e.g., at most about 80 wt %, at most about 75 wt %, at most about 70 wt %, at most about 65 wt %, at most about 60 wt %, at most about 55 wt %, or at most about 50 wt %) the solid content of the curable compositions described herein.

In some embodiments, the at least one filler described herein can include a first filler containing hollow silica particles and a second filler different from the hollow silica particles, such as a solid or hollow filler containing boron nitride, barium titanate, barium strontium titanate, titanium oxide, silica, (e.g., hollow glass), a fluoro-containing polymer (e.g., polytetrafluoroethylene), or silicone.

Examples of hollow and solid silicas include, but are not limited to: HS-200-TM (trimethylsilyl bonded), HS-200-MT (Methacrylsilyl bonded), HS-200-VN (Vinylsilyl bonded) and HS-200 (untreated) from AGC Si-Tech Co., Ltd. iM16K is hollow silica without surface modification and is available from 3M. SC2500-SVJ is a solid silica with surface modification and is available from Admatechs Co. Ltd. L250550 is a solid silica with surface modification and is available from 3M Company. The characteristics of the silica materials described above are summarized in Table 1 below.

TABLE 1

| | | | | Size | | SiO₂ | | | |
|---|---|---|---|---|---|---|---|---|---|
| Product | | Material | Surface | D₅₀ | Dₘₐₓ | content | Density | | |
| Name | Structure | Type | Modification | (μm) | (μm) | (wt %) | (g/mL) | Dk | Df |
| HS-200 | Hollow | Amorphous | No | 2 | <10 | >99% | 0.5-0.6 | 1.5-1.6 | 0.001 |
| | | Silica | | | | | | | |
| HS-200-MT | Hollow | Amorphous | Methacrylate | 2 | <10 | >99% | 0.5-0.6 | 1.5-1.6 | 0.001 |
| | | Silica | silane | | | | | | |
| HS-200-VN | Hollow | Amorphous | Vinyl silane | 2 | <10 | >99% | 0.5-0.6 | 1.5-1.6 | 0.001 |
| | | Silica | | | | | | | |
| HS-200-TM | Hollow | Amorphous | Trimethyl | 2 | <10 | >99% | 0.5-0.6 | 1.5-1.6 | 0.001 |
| | | Silica | silane | | | | | | |
| iM16K | Hollow | Soda-lime- | No | 20 | >20 | <90% | 0.5 | 1.5 | 0.005 |
| | | borosilicate | | | | | | | |
| | | glass | | | | | | | |
| SC2500- | Solid | Amorphous | Vinyl silane | 0.5 | <10 | >99% | 2.2 | 4 | 0.001 |
| SVJ | | Silica | | | | | | | |
| L250550 | Hollow | Soda-lime- | Vinyl silane | 19 | >20 | <90% | 0.5 | 1.5 | 0.005 |
| | | borosilicate | | | | | | | |
| | | glass | | | | | | | |
| Cellspheres | Hollow | Alumina | No | 3.8 | >10 | <90% | 0.6 | 1.8 | 0.003 |
| CS-NF | | borosilicate | | | | | | | |
| | | glass | | | | | | | |

D₅₀ = median particle diameter
Dₘₐₓ = maximum particle diameter

In some embodiments, the curable compositions described herein can include at least one (e.g., two or three or more) radical initiator (or free radical initiator). In some embodiments, the radical initiator generates free radicals at a temperature at least about 150° C. In some embodiments, the radical initiator can include a peroxide (e.g., di-(tert-butylperoxyisopropyl)benzene, bis(1-methyl-1-phenylethyl) peroxide, 2,5-dimethyl-2,5-di(t-butylperoxy)hexyne-3, 2,5-dimethyl-2,5-di(t-butylperoxy)hexane, dicumyl peroxide, dibenzoyl peroxide, dilauroyl peroxide, acetyl acetone peroxide, methyl ethyl ketone peroxide, methyl isobutyl ketone peroxide, bis(4-tert-butylcyclohexyl) peroxydicarbonate, dimyristyl peroxydicarbonate, tert-butyl peroxybenzoate, tert-butyl peroxy-2-ethylhexanoate, tert-butylperoxy 2-ethylhexyl carbonate, tert-butylperoxy isopropyl carbonate, tert-butyl peroxy-3,5,5-trimethylhexanoate, or 1,1-bis(tert-butylperoxy)-3,3,5-trimethylcyclohexane), an aromatic hydrocarbon (e.g., 1,1′-(1,1,2,2-tetramethylethylene)dibenzene, poly(1,4-diisopropylbenzene), 3,4-dimethyl-3,4-diphenyl hexane, or 2,3-dimethyl-2,3-diphenyl butane), or an azo compound (e.g., 2,2′-azobis(2-methylpropionitrile), azobisisobutyronitrile (AIBN), 1,1′-azodicyclohexanecarbonitrile, 2,2′-azodi(2-methylbutyronitrile), or 2,2′-azobis(2,4,4-trimethylpentane)). Without wishing to be bound by theory, it is believed the radical initiator can facilitate the curing of a curable composition (e.g., by initiating a cross-linking reaction of the low dielectric loss polymer described herein) when the composition is used to form a dielectric film.

In some embodiments, the radical initiator described herein is a non-peroxide radical initiator. In such embodiments, the radical initiator can be a hydrocarbon that contains no element other than carbon and hydrogen. In some embodiments, the hydrocarbon radical initiator can be aliphatic or aromatic, or can include both aliphatic and aromatic groups. Without wishing to be bound by theory, it is believed that decomposition products of peroxide initiators (and the peroxide initiators themselves) are polar and can lead to increased Df in cured compositions. On the other hand, without wishing to be bound by theory, it is believed that non-peroxide initiators (e.g., hydrocarbons such as 2,3-dimethyl-2,3-diphenyl butane or diazo initiators) do not contain polar atoms and therefore can lower the Df of cured compositions.

In some embodiments, the curable compositions described herein can include both a peroxide radical initiator and a non-peroxide radical initiator. Without wishing to be bound by theory, it is believed that a non-peroxide initiator may have a relatively high activation energy and therefore may not be efficient in curing the compositions described herein. Thus, without wishing to be bound by theory, it is believed that, in some embodiments, using a combination of a peroxide initiator and a non-peroxide initiator can result in a curable composition having optimal curing rate and Df.

In some embodiments, the curable compositions described herein do not include a radical initiator. In such embodiments, the compositions can be cured by heating.

In some embodiments, the radical initiator is present in an amount of from at least about 0.1 wt % (e.g., at least about 0.2 wt %, at least about 0.4 wt %, at least about 0.5 wt %, at least about 0.6 wt %, at least about 0.8 wt %, at least about 1 wt %, at least about 1.5 wt %, at least about 2 wt %, at least about 2.5 wt %, or at least about 3 wt %) to at most about 10 wt % (e.g., at most about 9 wt %, at most about 8 wt %, at most about 7 wt %, at most about 6 wt %, at most about 5 wt %, at most about 4 wt %, at most about 3 wt %, at most about 2.5 wt %, at most about 2 wt %, at most about 1.5 wt %, at most about 1 wt %, at most about 0.8 wt %, at most about 0.6 wt %, or at most about 0.5 wt %) of the solid content of the curable compositions described herein.

In some embodiments, the curable compositions described herein can optionally further include at least one (e.g., two or three or more) coupling agent. In some embodiments, the coupling agent can include a silane, a siloxane, a titanate, or a zirconate. In some embodiments, the coupling agent can include a reactive functional group, such as epoxy, cyanate ester, acrylate, methacrylate, amino, allyloxy, vinyl, and allyl groups. Examples of suitable coupling agents include diethoxymethylvinylsilane, trimethoxy(7-octen-1-yl)silane, octyltriethoxysilane, allyltrimethoxysilane, methacryloxypropyl-trimethoxysilane, vinyltrimethoxysilane, triethoxyvinylsilane, hydrolyzed vinylbenzylaminoethylamino-propyltrimethoxy silane, phenyltrimethoxysilane, (p-methylphenyl)trimethoxysilane, p-styryltrimethoxy-silane, amino ethyl amino triethoxy (or trimethoxy) silane, amino ethyl amino propyl triethoxy (or trimethoxy) silane, 3-isocyanatepropyltriethoxysilane, 3-methacryloxypropyl trimethoxysilane, tetra(2,2-diallyloxymethyl-1-butyl)bis(ditridecylphosphite)titanate, or tetra(2,2-diallyloxymethyl-1-butyl)bis(ditridecylphosphite)zirconate.

Other coupling agents envisioned include polysiloxanes (e.g., polyvinylsiloxanes, polyallylsiloxanes, and copolymers thereof), and polysilsesquioxanes (e.g., open or closed cage types of polysilsesquioxanes), allyl or vinyl silsesquioxanes. The coupling agent could also contain a fluorine atom. Tridecafluoro-1,1,2,2-tetrahydrooctyl(triethoxy) silane is an example. Coupling agents with long carbon chain aliphatic groups have the benefit of imparting flexibility to the resin composition. In some embodiments, the coupling agent can serve as a binding agent or an adhesive agent between two components in a curable composition or between a component in a curable composition and a surface (e.g., a copper surface) to which the curable composition is applied. Without wishing to be bound by theory, it is believed that the coupling agent can improve the dispersity of an inorganic filler in a curable composition, improve the adhesion between fillers and polymers in a curable composition, improve the adhesion between a substrate and polymers in a curable composition, improve the moisture and solvent resistance of a curable composition, and decrease the number of voids in a curable composition.

In some embodiments, the coupling agent can be applied onto the surface of the filler (e.g., as a surface treatment agent) before the filler is included in the curable compositions described herein. For example, the hollow silica can be treated with any one or combination of the following silanes: methacrylate, vinyl, epoxy, phenyl, decyl, dodecyl, n-octyl, tripropyl, aminopropyl, styryl, or linear partially fluorinated alkyl silanes having from 1-12 carbon atoms (tridecafluoro-1,1,2,2-tetrahydrooctyl)triethoxysilane for example) or polymethylsilsesquioxane. In some embodiments, the coupling agent can be included in the curable compositions described herein as a component independent of the filler. The same silanes can be added to the curable composition apart from the pretreatment to the filler surface.

In some embodiments, the coupling agent is present in an amount of from at least about 0.1 wt % (e.g., at least about 0.2 wt %, at least about 0.3 wt %, at least about 0.4 wt %, at least about 0.5 wt %, at least about 0.6 wt %, at least about 0.7 wt %, at least about 0.8 wt %, at least about 0.9 wt %, at least about 1 wt %, at least about 1.5 wt %, or at least about 2 wt %) to at most about 10 wt % (e.g., at most about 9 wt %, at most about 8 wt %, at most about 7 wt %, at most about 6 wt %, at most about 5 wt %, at most about 4 wt %, at most about 3 wt %, at most about 2 wt %, or at most about 1 wt %) of the solid content of the curable compositions described herein.

In some embodiments, the curable compositions described herein can optionally further include at least one (e.g., two or three or more) flame retardant. In some embodiments, the flame retardant can include one or more functional groups (e.g., vinyl, allyl, styryl, maleimide, citraconimide, or (meth)acrylate groups) capable of reacting with the low dielectric loss polymer described herein. In some embodiments, the flame retardant can be non-reactive to the low dielectric loss polymer described herein.

In some embodiments, the flame retardant described herein is free of halogen (e.g., F, Cl, Br, or I). In some embodiments, the flame retardant can include a phosphorus atom in its chemical structure. Examples of phosphorus-containing flame retardants include phosphate ester flame retardants, phosphinate flame retardants, and phosphazene flame retardants. Specific examples of suitable phosphorus-containing flame retardants include triphenyl phosphate, tricresyl phosphate, bisphenol A diphenylphosphate, aluminum diethylphosphinate (e.g., OP-935 available from Clariant Specialty Chemicals), p-xylylene-bis-diphenylphosphine oxide (e.g., PQ-60 available from Chin Yee Chemical Industries Co. Ltd.), (2,5-diallyloxyphenyl)diphenylphosphine oxide, hexaphenoxy cyclotriphosphazene (e.g., SPB-100 available from Otsuka Chemical Co. Ltd.), tris(2-allylphenoxy)triphenoxy cyclotriphosphazene (e.g., SPV-100 available from Otsuka Chemical Co. Ltd.), resorcinol bis(diphenylphosphate), resorcinol bis(di-2,6-dimethylphenyl phosphate) (e.g., PX-200 Daihachi Chemical Industry Co. Ltd.), diphenylphosphoryl){p-[(diphenylphosphoryl)methyl]phenyl}methane (e.g., BES5-1150 available from Regina Electronic Materials), cyanophenoxy(phenoxy) cyclophosphazenes, cresyloxy(phenoxy) cyclophosphazenes, spirocyclophosphazenes, benzylphenoxy cyclotriphosphazenes, allyl-containing phosphazenes, vinyl-containing phosphazenes, vinyl phenoxyphosphazenes (Rabitle FP-700 TP from Fushimi) phosphine oxides (e.g., bis(2,4,6-trimethylbenzoyl)phenylphosphine oxide, 2,5-diallyoxyphenyl)diphenylphosphine oxide, or [2,5-bis(4-ethenylphenylmethoxy)-phenyl]diphenylphosphine oxide), phosphites (e.g., tris(2,4-di-tert-butylphenyl)-phosphite, or melamine polyphosphates), acrylate functionalized organo phosphorous compounds (e.g., methacryloyloxymethyl diphenylphosphite oxide), 10-benzyl-9,10-dihydro-9-oxo-10-lambda(5)-phosphaphenanthrene-10-oxide. Without wishing to be bound by theory, it is believed that a phosphorus-containing flame retardant can lead to increased char formation and flame resistance, which can reduce the amount of the flame retardant and improve the properties (e.g., copper peel, ILBS, CTE, moisture resistance, and Df) of the curable composition.

In some embodiments, the flame retardant can be a halogen-containing flame retardant (e.g., a brominated flame retardant). Examples of such flame retardants include 1,1′-(ethane-1,2-diyl)bis(pentabromobenzene), N,N-ethylene-bis(3,4,5,6-tetrabromophthalimide), brominated polystyrenes, brominated polycarbonates, and hexabromocyclododecane.

In some embodiments, the flame retardant described herein can have a relatively low dissipation factor (Df). For example, the flame retardant can have a Df ranging from at most about 0.005 (e.g., at most about 0.0045, at most about 0.004, at most about 0.0035, at most about 0.003, at most about 0.0025, at most about 0.0022, at most about 0.002, at most about 0.0018, at most about 0.0016, at most about 0.0015, at most about 0.0014, at most about 0.0012, at most about 0.001, or at most about 0.0008) to at least about 0.0005 (e.g., at least about 0.0006, at least about 0.0008, or at least about 0.001).

In some embodiments, the curable compositions described herein can include an insoluble flame retardant (e.g., having a solubility at most about 1 mg/mL in an organic solvent). In some embodiments, the insoluble flame retardant can be uniformly dispersed in a curable composition.

In some embodiments, the curable compositions described herein can include a first flame retardant and a second flame retardant different from the first flame retardant. In some embodiments, the first flame retardant can be insoluble (e.g., having a solubility at most about 1 mg/mL) in an organic solvent, can be treated as an insoluble filler, and the second flame retardant can be soluble (e.g., having a solubility at least about 2 mg/mL) in an organic solvent. In some embodiments, the weight ratio of the first flame retardant to the second flame retardant is from at least about 0.5:1 (e.g., at least about 0.7:1, at least about 0.9:1, at least about 1.2:1, at least about 1.4:1, at least about 1.5:1, at least about 1.6:1, at least about 1.8:1, or at least about 2:1) to at most about 9:1 (e.g., at most about 4:1, at most about 3:1, at most about 2.5:1, at most about 2.4:1, at most about 2.2:1, or at most about 2:1). Without wishing to be bound by theory, it is believed that, although insoluble components (e.g., insoluble flame retardants) may have a relatively low Df, they can have inconsistent flame retardance and more difficulty passing a UL94 VO rating. Without wishing to be bound by theory, it is believed that using a combination of an insoluble flame retardant and a soluble flame retardant in a curable composition can result in a film having a consistent flame resistance and a relatively low Df.

In some embodiments, the flame retardant is present in an amount of from at least about 1 wt % (e.g., at least about 2 wt %, at least about 4 wt %, at least about 5 wt %, at least about 6 wt %, at least about 8 wt %, at least about 10 wt %, at least about 12 wt %, at least about 14 wt %, at least about 15 wt %, at least about 16 wt %, at least about 18 wt %, or at least about 20 wt %) to at most about 50 wt % (e.g., at most about 47 wt %, at most about 45 wt %, at most about 43 wt %, at most about 41 wt %, at most about 39 wt %, at most about 37 wt %, at most about 35 wt %, at most about 33 wt %, at most about 30 wt %, at most about 25 wt %, at most about 20 wt %, at most about 18 wt %, at most about 16 wt %, at most about 15 wt %, at most about 14 wt %, at most about 12 wt %, or at most about 10 wt %) of the solid content of the curable compositions described herein.

In some embodiments, the curable compositions described herein can optionally further include at least one (e.g., two or three or more) cross-linking agent. In some embodiments, the cross-linking agent can include triallyl isocyanurate, triallyl cyanurate, trimethylallyl isocyanurate, a bis(vinylphenyl)ether, bis(4-vinylphenyl)ethane, a bromostyrene (e.g., a dibromostyrene), a polybutadiene, a poly(butadiene-co-styrene) copolymer, divinylbenzene, a di(meth)acrylate, bisphenol A diallyl ether, acenapthylene, a cyanate ester, a maleimide compound (e.g., a bismaleimide), a dicyclopentadiene, a tricyclopentadiene, benzoxazines (e.g., allyl-containing benzoxazines or bisphenol A benzoxazines), phosphazenes (e.g., allyl-containing phosphazenes), allyl-containing cyclophosphazenes (e.g., tris(2-allylphenoxy)triphenoxy cyclotriphosphazene), 2,4-diphenyl-4-methyl-1-pentene, trans-stilbene, 5-vinyl-2-norbornene, tricyclopentadiene, dimethano-1H-benz[f]indene, 1,1-diphenylethylene, 4-benzhydrylstyrene, diisopropenylbenzene, diallylisophthalate, alpha-methylstyrene, a bis(vinylphenyl)ethane compound (e.g., 1,2-bis(4-vinylphenyl)ethane, 1,2-bis(3-vinylphenyl-4-vinylphenyl)ethane, 1,2-bis(3-vinylphenyl)ethane), a silane (e.g., a vinylsilane or allysilane), a siloxane (e.g., a vinylsiloxane or allysiloxane), or a silsesquioxane (e.g., a vinyl silsesquioxane or allyl silsesquioxane). Without wishing to be bound by theory, it is believed the cross-linking agent can facilitate the curing of a curable composition when the composition is used to form a dielectric film. Another example of a suitable crosslinking agent is a triazine derivative (L-DAIC) offered by Shikoku Kasei represented by the following:

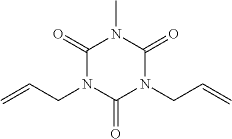 [SMILES: C=CCn1c(=O)n(C)c(=O)n(CC=C)c1=O]

The curable compositions can further contain cross-linkable polymers that crosslink with heat through chain transfer such a poly(methylstyrene) and crosslink through conventional free radical polymerization. Examples of the latter are: poly(styrene-co-divinylbenzene-co-ethylstyrene) copolymer and a poly(methylstyrene-co-4-(dimethylvinylsilylmethyl)styrene) copolymer.

In some embodiments, the cross-linking agent described herein is present in an amount of from at least about 1 wt % (e.g., at least about 1.5 wt %, at least about 2 wt %, at least about 3 wt %, at least about 4 wt %, at least about 5 wt %, at least about 6 wt %, at least about 7 wt %, at least about 8 wt %, at least about 9 wt %, or at least about 10 wt %) to at most about 20 wt % (e.g., at most about 18 wt %, at most about 16 wt %, at most about 15 wt %, at most about 14 wt %, at most about 12 wt %, at most about 10 wt %, at most about 8 wt %, at most about 6 wt %, at most about 5 wt %, or at most about 4 wt %) of the solid content of the curable compositions described herein.

In some embodiments, the curable compositions described herein can optionally further include at least one (e.g., two or three or more) organic solvent. A suitable organic solvent can be any solvent capable of mixing, dispersing, or dissolving the components of a curable composition. Preferably, the organic solvent can be completely or almost completely evaporated away during the coating or casting process at 250-350° F. such that the other components of a curable composition are the only components remaining on a carrier substrate. In some embodiments, the organic solvent includes tetrahydrofuran, acetonitrile, dimethylformamide, N-methylpyrrolidone, dimethylacetamide, acetone, 2-heptanone, methyl ethyl ketone, methyl isobutyl ketone, methyl n-amyl ketone, methyl isoamyl ketone, cyclopentanone, cyclohexanone, benzene, anisole, toluene, 1,3,5-trimethylbenzene, xylene, propylene glycol monomethyl ether, propylene glycol monomethyl ether acetate, or a combination thereof.

In some embodiments, the organic solvent is present in an amount of from at least about 1 wt % (e.g., at least about 5 wt %, at least about 10 wt %, at least about 15 wt %, at least about 20 wt %, at least about 25 wt %, at least about 30 wt %, at least about 35 wt %, or at least about 40 wt %) to at most about 50 wt % (e.g., at most about 45 wt %, at most about 40 wt %, at most about 35 wt %, at most about 30 wt %, at most about 25 wt %, at most about 20 wt %, at most about 15 wt %, or at most about 10 wt %) of the total weight of the curable compositions described herein. Without wishing to be bound by theory, it is believed that, if the organic solvent is less than about 1 wt % of a curable composition, the viscosity of the curable composition may be too high such that the curable composition may be not processed easily. In addition, without wishing to be bound by theory, it is believed that, if the organic solvent is more than about 50 wt % of a curable composition, in the absence of thixotropic thickening agents, the viscosity of the curable composition may be too low to keep the coated composition on a surface of a substrate due to non-wetting, which can lower coating uniformity and coating efficiency.

The curable compositions described herein can be prepared by methods well known in the art. For example, the curable compositions can be prepared by mixing the components together.

In some embodiments, the present disclosure features a film (e.g., a free-standing film or a supported film) prepared from a curable composition described herein. For example, a supported film can be prepared by extruding or coating a curable composition on a substrate to form a film supported by the substrate. As another example, a free-standing film can be prepared by coating a curable composition on a substrate to form a layer (e.g., a polymeric layer) and removing (e.g., peeling) the layer from the substrate to form the free-standing film. In some embodiments, the film (e.g., a free-standing or supported film) is partially cured. In some embodiments, the film (e.g., a free-standing or supported film) is not cured. In some embodiments, the film described herein can be a dielectric film.

In some embodiments, a film formed by a curable composition described herein has a dielectric constant (Dk) of from at most about 3.5 (e.g., at most about 3.2, at most about 2.9, at most about 2.8, at most about 2.7, at most about 2.6, at most about 2.5, at most about 2.4, at most about 2.2, or at most about 2) to at least about 1.5 (e.g., at least about 1.8) at 10 GHz. In some embodiments, a film formed by a curable composition described herein has a dissipation factor (Df) of from at most about 0.004 (e.g., at most about 0.0035, at most about 0.003, at most about 0.0025, at most about 0.002, at most about 0.0018, at most about 0.0016, at most about 0.0015, at most about 0.0014, at most about 0.0012, at most about 0.001, or at most about 0.0008) to at least about 0.0005 (e.g., at least about 0.0006, at least about 0.0008, at least about 0.001, or at least about 0.002) at 10 GHz.

In some embodiments, a film formed by a curable composition described herein has a tensile modulus of from at most about 15000 MPa (e.g., at most about 10000 MPa, at most about 7000 MPa, at most about 5000 MPa, at most about 3000 MPa, at most about 2500 MPa, at most about 2000 MPa, at most about 1500 MPa, at most about 1000 MPa, at most about 500 MPa, at most about 100 MPa, or at most about 50 MPa) to at least about 1 MPa (e.g., at least about 10 MPa, at least about 100 MPa, or at least about 500 MPa).

In some embodiments, the present disclosure features a build-up film that includes a number of layers (e.g., 10-15 layers), in which at least one (e.g., two, three, four, or more) layer or each layer is prepared from a curable composition described herein. For example, the present disclosure features a multilayer circuitry having a build-up film described herein, in which stacked microvias can be disposed over each other and are electrically connected. In some embodiments, the build-up film or multilayer circuitry can have improved dimensional stability when it is subject to repeated heating and cooling cycles from room temperature to 260° C.

In some embodiments, the present disclosure features an article that includes a carrier and at least one layer supported by the carrier, in which the at least one layer includes a film described herein. In general, the carrier is dimensionally stable and resistant to the solvent and temperature used in a coating process. In some embodiments, the carrier can include a metal foil, paper, or a polymer. For example, the metal foil can be a stainless steel foil, a copper foil, a copper foil comprised of a microthin copper and a heavier supporting copper layer, an aluminum foil, or an aluminum foil treated with palladium. As another example, a carrier including a polymer can be a polymer coated thermoplastic film (e.g., a film made from polyimide, polyethylene naphthalate (PEN), polyethylene terephthalate (PET), release coated PET, a liquid crystal polymer (LCP), polyetherimide, polytetrafluoroethylene (PTFE), polyether ether ketone (PEEK), or polybenzimidazole), or a polymer coated non-reinforced material (e.g., paper). In some embodiments, the carrier described herein can include a release coating (e.g., a fluorocarbon, a polymeric silicone resin, a silicone oil, polyethylene, polyvinyl chloride, or a polyester) to adjust its release properties. In some embodiments, the carrier includes a non-woven substrate (e.g., paper). In general, the carrier can have a curable composition coated on one surface or on both surfaces.

In some embodiments, the present disclosure features an article comprising: a fiberglass based substrate core, a glass core with or without metallized through vias, or a TSV; and from 1 to 10 build up film layers that are laminated on top, bottom or both sides of the substrate core, glass core, or TSV, together with copper metallization such that circuitry are created that is flame retardant and passes UL VO.

In some embodiments, a curable composition coated carrier can be transferred (e.g., by hot roll lamination) to a surface of another substrate (e.g., a printed wiring board surface containing electronic circuitry) while the carrier film is intact. After the curable composition is transferred, the carrier can be removed to enable further printing wiring board (PWB) manufacturing. In some embodiments, the curable composition described herein can be laminated to a copper substrate followed by printing and etching to create copper lines and traces or copper artwork typical of PWB manufacturing. In the case of a microthin copper bonded to a thicker, support copper layer, after hot roll lamination of the coppers or hydraulic high temperature lamination of the microthin layer to the substrate, the support copper can be removed after which the microthin layer can be further processed to create copper circuitry.

In some embodiments, the curable composition can be cured at the surface of the printed wiring board by heating in an oven at the cure temperature in an inert environment. The dielectric surface thus obtained can then be slightly or aggressively desmeared with permanganate or plasma to etch some resin from around the filler particles present in the curable dielectric composition. Palladium can then be deposited into the dielectric material as a seed layer for depositing copper plating. This is known as electrolysis copper plating. Once a layer of electroless copper plating has been added to the dielectric surface, additional copper can be added by electrolytic copper plating. This process has the advantage that a photoresist layer can be coated or laminated onto the surface of the PWB before the use of electrolytic copper. The resist layer can be imaged such that only the areas where further copper plating is desired are exposed. The photoresist is opened up where signal traces are desired or where the plating of copper through holes is desired. Electrolytic plating then only occurs in a vertical direction. This is known as the semi-additive process (SAP). Once the resist is stripped, the very thin layer of electroless copper can be removed by etching. The SAP process allows very fine lines and spaces because the fine lines and spaces are created by vertically building the copper up as opposed to a subtractive process where the layers of coppers are etched away and the copper features are created by selective etching of copper in certain areas. The palladium can be deposited into the dielectric material by using a conventional electroless copper plating technique. A nanoparticle suspension of palladium particles can be employed that appears as a palladium ink. The palladium ink has a feature that the nanoparticle suspension can penetrate into very fine openings in the dielectric. The peel adhesion strength of the resulting built up copper can be higher than that made from conventional technologies. This is referred to in the industry as the A-SAP process.

In some embodiments, a curable composition described herein can be applied to a surface of a metal substrate (e.g., a copper foil) to form a resin coated metal substrate (e.g., a resin coated copper (RCC) foil) in which the metal substrate serves as a carrier. In such a resin coated metal substrate, the curable composition can be either cured (e.g., partially or fully) or uncured. In some embodiments, the metal substrate (e.g., copper or aluminum) can include a thin layer of palladium. In some embodiments, the copper foil can be ⅓ oz copper foil, % oz copper foil, 0.5 oz copper foil, or 1 oz copper foil. The resin coated metal substrate can be used as a dielectric material for building multilayer circuitry in printed circuit boards (PCBs) or wiring boards (PWBs). For example, a resin coated copper foil can be laminated onto the surface of a PWB in a process known as a foil lamination. The resulting composite can have a layered structure consisting of a copper foil, a dielectric resin layer, and a PWB having features such as high density interconnect (HDI) layers, copper lines and traces, and mechanical through vias. After the resin coated copper has been laminated to the surface of the PWB and cured, the PWB can be further fabricated by selectively etching/removing copper in the areas desired to create copper features.

In some embodiments, an alternative approach to form a RCC is to coat micro thin copper that is ultrasonically bonded, chemically bonded or bonded in some fashion to a much heavier carrier copper. The bonding of the micro thin copper to the heavier carrier copper could be achieved by using a thin layer of electroless copper that bonding the micro thin copper and heavier carrier copper together. For example, 1.5 μm and 3.0 μm thin copper can be bonded to a much heavier 0.5 or 1.0 oz carrier copper. Examples of such bonded copper include MT18FL, MT18X and MT18GN from Mitsui Kinzoku. Similar micro thin copper bound to a carrier copper include DoubleThin™ NN, ANP, and NF having a carrier copper of 0.5 and 1.0 oz, which are available from Circuit Foil.

In some embodiments, the micro thin copper is designed to be released from the carrier copper during lamination. For example, the curable composition described herein can be deposited onto the face of the two layered copper containing the micro thin copper to form a dielectric layer. After the dielectric layer is laminated to the surface of a PWB, the PWB has a structure that includes a sacrificial copper carrier (e.g., 0.5 oz copper carrier) disposed on a micron thin copper (e.g., having a thickness of 1.5 μm and 3.0 μm), which lies on top of a dielectric layer that further lies on top of a PWB circuitry. During lamination, the sacrificial copper carrier can be debonded and removed, leaving a surface of micro thin copper bonded to the dielectric layer which is further bonded to the surface of the printed wiring board. This process has the advantage that the micro thin copper can be subtractively etched or micro etched such that a very fine resolution of copper traces can be achieved. This process is known in the art as M-SAP. The very fine features of copper can then by electrolytically plated up to the desired thickness. In such a process, it is desirable for the dielectric layer to be both flexible and resistant to cracks, and have good adhesion to thin, low profile copper.

In some embodiments, the present disclosure features a prepreg product prepared from a curable composition described herein. In some embodiments, the prepreg product includes a base material (e.g., a woven or non-woven substrate (such as a fabric or a fibrous material)) impregnated with a curable composition described herein. The base material is also known as the supporting or reinforcing material. The prepreg products described herein can be used in the electronics industry, e.g., to produce printed wiring or circuit boards.

In general, the prepreg products described herein can be produced by impregnating a base material (usually based on glass fibers, either as a woven or nonwoven substrate or in the form of a cross-ply laminate of unidirectionally oriented parallel filaments) with a curable composition described herein, followed by curing the curable composition wholly or in part (e.g., at a temperature ranging from about 150° C. to about 250° C.). The base material impregnated with a partially or wholly cured composition is usually referred to as a “prepreg.” As mentioned herein, the terms “prepreg” and “prepreg product” are used interchangeably. In some embodiments, the base material can be impregnated by applying a dielectric film described herein above the base material, applying a dielectric film described herein below the base material, and pressing the dielectric films into the base material. To make a printed wiring board from a prepreg, one or more layers of the prepreg are laminated with, for example, one or more layers of copper.

In some embodiments, the base material (e.g., containing a woven or non-woven substrate) used in the prepregs described herein can include inorganic fiber base materials such as glass. A glass fiber base material is preferable from the viewpoint of flame resistance. Examples of the glass fiber base materials include, but are not limited to, woven fabrics using E glass, NE glass (from Nittobo, Japan), C glass, D glass, S glass, T glass, Quartz glass, L glass, L2 glass, or NER glass; glass non-woven fabrics in which short fibers are adhered into a sheet-like material with an organic binder; and those in which glass fiber and other fiber types are mixed and made fabric.

In some embodiments, a prepreg can be produced by impregnating a curable composition described herein into a base material (e.g., a woven or non-woven substrate) followed by drying. In another embodiment a pure dielectric film of the present invention can be suspended above and below a coated fiberglass or uncoated fiberglass base material such that the resin composition of the present invention can be tack bonded or hot roll laminated onto the based material with a degree of curing ranging from no cure to partial cure. In some embodiments, the prepregs described herein can have a resin content as defined herein of from at least about 30 wt % (e.g., at least about 40 wt %, at least about 50 wt %, at least about 60 wt %, at least about 65 wt %, at least about 70 wt %, at least about 75 wt %, at least about 80 wt %, at least about 85 wt %, or at least about 90 wt %) to at most about 90 wt % (e.g., at most about 80 wt %, at most about 70 wt %, at most about 65 wt %, at most about 55 wt %, at most about 50 wt %, at most about 45 wt %, at most about 40 wt %, at most about 35 wt %, or at most about 30 wt %). Without wishing to be bound by theory, it is believed that a prepreg having a relatively high resin content would have improved electrical properties, while a prepreg having a relatively low resin content would have improved thermal properties and an extremely low level of thermal expansion.

In some embodiments, a metal substrate can be applied to one or both surfaces of the prepreg thus formed to form a laminate. In some embodiments, the prepreg formed above can optionally be laminated with one or more layers of prepregs as necessary to make a composite structure, and a metal foil (e.g., a copper or aluminum foil) can be applied to one or both surfaces of the composite structure to obtain a laminate (or a metal clad laminate). The laminate thus formed can optionally be subjected to further treatment, such as pressurization and hot pressing, which can at least partially (or fully) cure the prepreg layers. The laminate (e.g., a copper clad laminate) can be further layered with additional prepreg layers and cured to make a multilayer printed circuit board.

In some embodiments, the present disclosure features a laminate that includes at least one (e.g., two or three or more) layer prepared from the prepreg product described herein. In some embodiments, the laminate can include (1) a copper substrate (e.g., a copper foil) and (2) at least one prepreg layer laminated on the copper substrate. In some embodiments, one or both surfaces of the prepreg layer can be laminated with the copper substrate. In some embodiments, the present disclosure features a multilayer laminate in which multiple copper clad laminates described herein are stacked on top of each other optionally with one or more prepreg layers between two copper clad laminates. The multilayer laminate thus formed can be pressed and cured to form a multilayer printed circuit board.

In some embodiments, the present disclosure features a laminate that includes first and second films or a plurality of films, and a woven or non-woven substrate (e.g., containing a glass cloth such as a fiberglass cloth) between the first and second films, in which at least one (e.g., both) of the first and second films is a dielectric film described herein (i.e., a dielectric film prepared from a curable composition described herein). In some embodiments, the substrate can be pretreated with another resin composition/film or can be free of any other resin composition/film. For example, woven fiberglass with no treatment other than a silane coupling agent can be an example of an untreated substrate. In some embodiments, the first and second films can have substantially the same thickness. Without wishing to be bound by theory, it is believed that, when a laminate has the same dielectric film with the same thickness on both sides of the substrate, the laminate can exhibit uniform electrical performance, tight thickness control, and improved flatness. In some embodiments, the first and second films can have different thicknesses. For example, in some embodiments, it is necessary to have a thick layer of resin composition to fill and flow into the cavities of a printed wiring board. In such embodiments, it can be desirable to have a dielectric film on one side of the substrate with a larger thickness than the dielectric film on the other side of the substrate such that the laminate is capable of fully encapsulating copper circuitry.

In some embodiments, the laminate described above can further include a metal foil (e.g., a copper foil) on a surface of the first or second film. In some embodiments, the laminate described herein can further include first and second metal foils (e.g., copper foils) to form a metal clad laminate (e.g., a copper clad laminate), in which the first film is between the first metal foil and the substrate and the second film is between the second metal foil and the substrate. In some embodiments, the present disclosure features a multilayer laminate in which multiple metal clad laminates described herein are stacked on top of each other. The multilayer laminate thus formed can be pressed and cured to form a multilayer printed circuit board.

In some embodiments, the present disclosure features a laminate that includes first and second metal foils (e.g., copper foils), and a dielectric film described herein (i.e., a dielectric film prepared from a curable composition described herein) between the first and second foils. For example, a curable composition described herein can be coated onto one or both sides of a carrier substrate (e.g., paper). The dielectric layer thus formed can then be removed from the carrier substrate after B staging and then laminated with a copper foil to create a non-reinforced copper clad laminate.

In some embodiments, a curable composition described herein can be coated onto a nodule treated side of a copper foil. The amount of the composition coated onto the nodule treated side of the copper depends on the desired thickness of the dielectric layer, which can range from 0.1 mil to 5 mils. After B staging, the resin coated copper could be combined with another sheet of copper foil to yield a copper clad laminate in which copper foils are on both sides of the dielectric layer. In some embodiments, two sheets of resin coated copper foils can be laminated together to yield a copper clad laminate that includes one combined dielectric layer between two copper foils. In some embodiments, a sheet of unclad non-reinforced cured dielectric resin sheet prepared from a curable composition described herein can be inserted between two RCC layers to form a dielectric layer having any suitable desired thickness.

In some embodiments, the laminate described herein has a dielectric constant (Dk) of from at most about 5.0 (e.g., at most about 4.0, at most about 3.5, at most about 3.0, at most about 2.5, at most about 2.2, or at most about 2.0) to at least about 1.5 at 10 GHz. In some embodiments, the laminate has a dissipation factor (Df) of from at most about 0.004 (e.g., at most about 0.0035, at most about 0.003, at most about 0.0025, at most about 0.002, at most about 0.0018, at most about 0.0016, at most about 0.0015, at most about 0.0014, at most about 0.0012, at most about 0.001, or at most about 0.0008) to at least about 0.0005 (e.g., at least about 0.0006, at least about 0.0008, or at least about 0.001) at 10 GHz. As mentioned herein, the Dk and Df of a dielectric film/metal laminate (e.g., a multilayer laminate) is measured after the metal layer is removed. Without wishing to be bound by theory, it is believed that laminate having relatively low Dk and/or Df can reduce the total dielectric loss and lower signal loss.

The printed circuit industry is constantly presented with greater need for densification. In general, densification requires increasingly thin dielectric layers. Increasingly thin dielectric layers can be realized from either HDI build up or increasingly thin copper clad laminates. Conventional thin dielectric layers are prone to cracks in all stages of the manufacturing process. However, the low modulus dielectric layers or films described herein are well suited to meet the densification demand as they are flexible when thin, compliant, and capable of some elongation without forming cracks. In addition, a non-reinforced dielectric film described herein can achieve dimensional stability of 0.5 to 0.6 mils/inch without any fiberglass reinforcement (IPC-650 2.2.4 [TS]).

In some embodiments, a dimensionally stable laminate can be achieved by placing a free standing dielectric resin film described herein above and below a piece of fiberglass such that the free standing film gets into intimate contact with the fiberglass. The fiberglass can be a flattened weave or an open weave used in electronics. The fiberglass can be made from E glass, lower DF/DK fiberglass, NE Glass, NER glass from Nittobo, L or L2 glass, quartz, or C, D, S, or T glass. The free standing film at the surface of a fiberglass can have the benefit of lower modulus and resistance to cracking or warping at the surface. In some embodiments, copper can be placed at both outer surfaces of the dielectric resin films. This approach to the preparation of a dimensionally stable laminate has the advantage that a heavier fiberglass with extremely low CTE can be combined with a very low CTE dielectric resin such that a copper clad laminate can be produced that has a CTE from 5-10 ppm/° C. meeting the requirements for stable materials for chip carriers or multichip modules. In some embodiments, it can be advantageous to dispose the free standing dielectric film above and below a piece of fiberglass that has already been pretreated with another resin system. These resins can include PPE, pyrimidine, cyanate ester, butadiene, triallylisocyanurate, bismaleimide, divinylbenzene, divinylbenzene/styrene copolymer, divinylbenzene/styrene, ethyl or methyl vinyl benzene copolymer, poly(styrene-butadiene), poly(SBS), SEBS, SEPS, SIS, partially or fully hydrogenated versions thereof, and methyl or maleic anhydride functionalized versions thereof. In general, it is difficult to coat or impregnate a fiberglass substrate with a resin coating on both sides with high levels of solids and good thickness control. Without wishing to be bound by theory, it is believed that disposing a dielectric film described herein above and below a fiberglass substrate or a non-woven substrate can meet these requirements and ensure that the dielectric spacing is consistent on both sides.

The laminates can be made from the curable compositions disclosed herein by suitable methods known in the art. For example, copper clad laminates can be manufactured by laminating from 375 to 480° F. In general, the amount of the curable composition should be adjusted during lamination to generate uniform laminates with no voids. In some embodiments, the amount of flow during lamination can be increased by the following methods: using lower viscosity or lower molecular weight versions of the existing raw materials, increasing the composition of the low molecular weight components such as the catalyst, incorporating low viscosity small molecules capable of cross linking such as bis(4-vinylhenyl)ethane (BVPE) from Regina Electronic Materials, bisphenol A diallyl ether from Evonik, allyl substituted triazines, small molecule monofunctional benzoxazines, and acenaphthylene. The amount of flow during lamination can be reduced by increasing the amount of B-staging, or using higher viscosity or higher molecular weight version of the existing raw materials. In some embodiments, flow can be reduced by the proper choice of a flexible elastomer such as a SEBS or SEPS polymer. Particle size and shape of the filler components can also act as flow inhibitors. Hot roll lamination and vacuum lamination are best achieved with low viscosity curable compositions. It is preferred that the curable composition achieve a minimum flow viscosity from 100-20,000 poise, preferably from 100-10,000 poise.

In general, the increasing densification of circuitry requires smaller copper lines and spaces, such as 10-25 microns (e.g., 5 to 10 microns). Thus, resin coated copper or dielectric layers of 0.5 to 1 mil in thickness is desired. In addition, it is preferable that a resin coated copper or dielectric layer is defect free. Thus, in some embodiments, any inorganic or organic particles added to the curable composition described herein is at most about 10 microns (e.g., at most about 5 microns). In some embodiments, all insoluble particulates in the curable composition described herein can be dispersible in the solvent of choice, can be filtered if necessary in recirculation processes, can be processed without particle agglomerates causing defects in the consolidated resin film. In general, large particulates can be a scrap defect in manufacturing when the large particulates prevent the proper coating or extrusion to deposit the curable composition. The resulting defects can be voids, can be a large concentration of a single non-uniform component, or can lead to uneven composition across the width of the manufacturing equipment. In particular, the following defects can be caused by large particulates: laser hole irregularity, drilled hole irregularity, shorts, microshorts, inconsistent flame resistance, inconsistent coefficient of thermal expansion where a resin rich area cracks and a particle rich region does not, voids caused by agglomerates resisting or prohibiting resin flow, solder shock failures due to inconsistent CTE values, uptake of unwanted process chemistry due to high concentrations of particulates that lead to unwanted porosity. In addition, large concentrations of an undispersed or non-dissolved particle can lead to irregular flow or irregular curing of the resin composition. The above defects can result in non-uniform dielectric properties.

In some embodiments, all insoluble components that are present in the curable compositions described herein can have a particle size of at most about microns. The size of an insoluble component can be reduced by methods known in the art. For example, the particle size of a component can be reduced by milling in a dry or wet process. In general, dry milling does not involve a solvent or dispersing in water. In some embodiments, when larger particles are only a small fraction of an insoluble component, an air classifier can be used to remove larger particles. An air classifying mill can be used to simultaneously grind particulates to reduce their size and remove particulates larger than a certain diameter. In some embodiments, an air classification mill can be a two stage mill where large particulates enter the grinder and do not leave the air classifying mill until a desired particle sized is reached. Dry milling can typically reduce particles to a diameter of at most about 2-3 microns. Further particle size reduction can be achieved by using ball mill, attrition mill, sand mill, or small media mill. For example, particles are broken down by the size of the balls used in the ball mill to shear the particles between the media particles. A small media mill uses much smaller beads (e.g., having a diameter of 0.1 to 1 mm) to break the particles down even further reaching the submicron sizes. In some embodiments, attrition mills use media 3-10 mm in diameter whereas ball mills use media having a diameter of at least about 20 microns. Any such particle size reduction technique could be used with or without solvent or water. Adding a fluid can help control heat buildup during the particle size reduction. The particulates can be any organic or inorganic component that is not soluble in the solvent used in a curable composition described herein.

In some embodiments, the present disclosure features a printed circuit or wiring board obtained from the laminate described herein. For example, the printed circuit or wiring board can be obtained by performing circuit processing on the copper foil of a copper foil clad laminated board. Circuit processing can be carried out by, for example, forming a resist pattern on the surface of the copper foil, removing unnecessary portions of the foil by etching, removing the resist pattern, forming the required through holes by drilling, again forming the resist pattern, plating to connect the through holes, and finally removing the resist pattern. A multi-layer printed circuit or wiring board can be obtained by additionally laminating the above copper foil clad laminated board on the surface of the printed wiring board obtained in the above manner under the same conditions as described above, followed by performing circuit processing in the same manner as described above. In this case, it is not always necessary to form through holes, and via holes may be formed in their place, or both can be formed. For example, in a printed circuit board (PCB), two pads in corresponding positions on different layers of the circuit board can be electrically connected by a via hole through the board, in which the via hole can be made conductive by electroplating. These laminated boards are then laminated the required number of times to form a printed circuit or wiring board.

The printed circuit or wiring board produced in the above manner can be laminated with a copper substrate on one or both surfaces in the form of an inner layer circuit board. This lamination molding is normally performed under heating and pressurization. A multi-layer printed circuit board can then be obtained by performing circuit processing in the same manner as described above on the resulting metal foil clad laminated board. The printed circuit board thus obtained can be used in an electronic product (e.g., a semiconductor chip).

In some embodiments, the build up films can be used in combination with glass core substrates. Semiconductor packaging is growing in size with the adoption of chiplets. Warpage is a key concern of large semiconductor packages. Generally speaking a substrate core or something that achieves the same result is used to control warpage of the semiconductor package. An alternative to a fiberglass based substrate core is a glass core. The key factors to preventing warpage of a semiconductor package is the choice of the substrate core or glass core to control warpage. To ensure flatness, the following key engineering values are critical in the substrate core or the glass core: the thickness of the substrate core or the glass core, the CTE values in X/Y, and the modulus. The build up film is also critical in helping to resist warpage of the semiconductor package. Low CTE and low modulus of the build up film is required. It is necessary that the build up film have some form of internal stress relief. Without being bound by theory, it is important that the build up film can be laminated onto either a substrate core, a TSV, or a glass core and have sufficient adhesion that sputtered copper or electroless copper can be deposited on the build up film and that the circuits are robust regardless of how fine the lines and spaces of circuitry that are created on the copper supported by the build up film. It is an embodiment that layers of build up film be sequentially laminated onto one or both sides of a substrate core layer, or a glass sheet layer, or a PDL layer, or a TSU layer, such that circuitry can be formed after each sequential lamination, such that any number of build up layers and their associated circuitry can be applied on one of both sides of another substrate material. It is an embodiment that the build up films are inherently flame retardant and do not rely on the particular properties of the other materials for flame retardancy.

### EXAMPLES

The present disclosure is illustrated in more detail with reference to the following examples, which are for illustrative purposes and should not be construed as limiting the scope of the present disclosure.

### Materials

In the Examples below, the following materials were used. CCDFB is a non-peroxide initiator (i.e., 2,3-dimethyl-2,3-diphenylbutane) available from United Initiators, Inc. Saytex-8010 is a bromine-containing flame retardant (i.e., 1,1′(ethane-1,2-diyl)bis[pentabromo-benzene]) available from Albemarle Corp. AX-32 is a spherical alumina available from Sibelco. SS-15V is a spherical silica available from Sibelco. Poly(S-1-S) is a styrene-isoprene-styrene copolymer (Vector 4411) available from Dexco Polymers. Poly(S-B-S) (SBS-A) is a styrene-butadiene copolymer rich in butadiene available from Nisso America Inc. Trilene 65 and 67 are ethylene propylene diene (EPDM) terpolymer resins available from Kraton Corporation. Kraton 1535H, 1536H, 1648 and D1623 are a SEBS available from Kraton Corporation. SLK1500 is a bismaleimide (BMI) available from Shinetsu and having the same structure as BMI-1500 described above. SLK3000 is an aliphatic bismaleimide available from Shinetsu and having the same structure as BMI-3000 described above. MIR5000 is a maleimide functionalized poly(arylisopropylidene) available from Nippon Kayaku. Septon 1020 is an SEPS elastomer available from Kuraray Co. Ltd. M1913 is an SEBS elastomer containing about 30 wt % styrene monomer unit and modified by maleic anhydride available from Asahi Kasei Corp. Septon V9461 is a hydrogenated methylstyrene functionalized elastomer available from Kuraray Co. Ltd. KR511 is a vinyl/phenyl siloxane coupling agent available from Shinetsu. OFS-6518 is a vinyltrimethoxysilane available from Dow, Inc. OFS-6030 is methacryloxypropyl trimethoxysilane available from Dow, Inc. BMI-689 is a bismaleimide containing long hydrocarbon chains available from Designer Molecules Inc. BMI-TMH is 1,6-bismaleimide (2,2,4-trimethyl)hexane available from Daiwakasei Industries. MDAB is 4,4′-bismaleimidodiphenyl methane available from Evonik Industries. MXBI is m-xylenebismaleimide available from Evonik Industries. EH-BMI-2M2E is bis(3-ethyl-5-methyl-4-maleimidophenyl)methane available from the Anwin Group. SA-9000 is a poly(phenylene ether) available from Sabic. JSR HC-21 resin is an arylether pyrimidine copolymer available from Japanese Synthetic Rubber Corporation. PPE/butadiene copolymer is available from Nippon Kayaku. SPV-100 is tris(2-allylphenoxy)triphenoxy cyclotriphosphazene available from Otsuka Chemical Co. Ltd. W-1o is (2,5-diallyloxyphenyl)diphenyl phosphine oxide available from Katayama Chemical. PQ-60 is p-xylylene-bis-diphenylphosphine oxide available from Chin Yee Chemical Industries Co. Ltd. FP-72TP is a spirocyclophosphazene available from Fushimi Pharmaceutical Corp. Fushimi FP300 is a cyanophenoxy phenoxy(cyclophosphazene). BES5-1150 is diphenylphosphoryl{p-[(diphenylphosphoryl)methyl]phenyl}-methane (also known as p-xylylene-bis-diphenylphosphine oxide) available from Regina Electronic Materials. Araldite MT 35610 is a bisphenol A benzoxazine available from Huntsman. P-d Benzoxazine and LDAIC are available from Shikoku Kasei.

### General Procedure 1

### Property Measurements

### Preparation of Unreinforced Films for Resin Flow

A curable composition was prepared by dissolving the soluble resin components in toluene. Insoluble silica and flame retardant components were added and dispersed in the resin varnish using a rotor-stator mixer. The resin composition was then coated onto a polymer release coated paper using a wire-wound wrapped iron Mayer wet film applicator rod to a consistent thickness. The resin composition on the paper carrier was then heated at 150° C. for ten minutes or until all toluene was evaporated. The consolidated resin composition was then separated from the release coated paper to yield an unreinforced film.

### Preparation of Unreinforced Laminates for Dk/Df and CTE Measurements

The above unreinforced film was then laminated between two pieces of 0.5 oz HS1 VSP copper foil to yield a double sided copper clad laminate using the below cycle I. Portions of the sample were etched to remove the copper for dielectric property measurements and other portions the copper were retained for copper peel strength measurements.

Cycle I: The laminate was heated from room temperature to 450° F. at a heating rate of 6° F./min, kept for 2 hours at 450° F., and cooled down to room temperature at a cooling rate of 10° F./min. The lamination pressure was around 50 psi.

### Dk and Df Tests

Df and Dk values were analyzed by using IPC TM-650 Method 2.5.5.13 Split Post Cavity (SPC) Measurement method. The Df and Dk values at 10 GHz were measured by Network Analyzer N5230A from Agilent Technologies.

### Cu Peel Strength Test

Cu peel strength was measured based on 0.5 or 1 oz Cu weight per unit area using the IPC-TM-650 TEST METHODS MANUAL 2.4.8. Peel Strength. United SSTM-1 Model was used for Cu peel strength measurement.

### Resin Flow

Flow of an unreinforced film was measured using the IPC-TM-650 TEST METHODS MANUAL 2.3.17.

### CTE Measurements

CTE (z-axis) values from 25° C. to 260° C. were measured by TMA 450 from TA Instruments using 0.25 inch by 0.25 inch of unreinforced laminates.

### Examples 1-38: Preparation and Characterization of Curable Compositions 1-38

Curable composition 1 was prepared using the following procedure: Toluene was first added to a mixing container to which is attached a high sheer mixer. The high viscosity resin was first added, followed by the low molecular weight or low viscosity fluids. Specifically, 2.2 parts of an ethylene propylene diene (EPDM) terpolymer resin (i.e., Trilene 67) was first dissolved in toluene, followed by 2 parts of a poly(S-1-S) copolymer (Vector 4411). To this stirred solution was added in the following order: 7.2 parts of a 1,2-polybutadiene resin (B3000 available from Nisso Chemical), 4.0 parts of a poly(S—B-S) (SBS-A), 2.8 parts of bisphenol A benzoxazine (Araldite MT 35610 available from Huntsman), and 0.4 parts of 2,3-dimethyl-2,3-diphenylbutane (CCDFB). The remaining components were added to the above mixture and were dispersed with a high shear Ross mixer in the following order: 1.7 parts 1,3-phenylbismaleimide, 49.4 parts of spherical silica (SS-15V), 22.3 parts of spherical alumina (AX-32), and 8 parts of the flame retardant 1,1′(ethane-1,2-diyl)bis(pentabromine) (Saytex 8010). Additional toluene was then added to adjust the specific gravity to approximately 1.5.

The resin composition obtained above was then coated onto a polymer release coated paper using a wire-wound wrapped iron Mayer wet film applicator rod to a consistent thickness. The resin composition on the paper carrier was then heated at 150° C. for ten minutes or until all toluene was evaporated. The consolidated resin composition was then separated from the release coated paper to yield a free standing film. The free standing film was then laminated between two pieces of 0.5 oz HS1 VSP copper foil to yield a double sided copper clad laminate. Portions of the sample were etched to remove the copper for dielectric property measurements and other portions the copper were retained for copper peel strength measurements.

Curable compositions 2-27, 30, and 33 were prepared and evaluated using the same procedures described above. Curable compositions 28, 29, 31, 32, and 34-38 are prophetic. Curable compositions 1-38 and their properties of curable compositions 1-27, 30, and 33 were summarized in Tables 2-5 below. The amount of each component in Tables 2-5 is part by weight.

TABLE 2

| Example | 1 | 2 | 3 | 4 | 5 | 6 | 7 | 8 |
|---|---|---|---|---|---|---|---|---|
| CCDFB | 0.4 | 0.4 | 0.4 | 0.4 | 0.4 | 0.4 | 0.4 | 0.4 |
| Saytex 8010 | 8 | 9.4 | 9.4 | 9.4 | 8 | 8 | 8 | 8 |
| Alumina AX-32 | 22.3 | 23.5 | 23.5 | 23.5 | 18.3 | 18.3 | 18.3 | 18.3 |
| Silica SS-15V | 49.4 | 47.1 | 47.1 | 47.1 | 53.4 | 53.4 | 53.4 | 53.4 |
| Poly(butadiene) | 7.2 | 8 | 8 | 8 | 8.1 | 8.1 | 4.2 | 4.2 |
| 1,3-Phenyl | 1.7 | | | 1.9 | | | 1.9 | 1.9 |
| bismaleimide | | | | | | | | |
| Poly(S—I—S) | 2 | | | | | | | |
| Bisphenol A | 2.8 | 3.1 | 3.1 | 3.1 | 3.2 | 3.2 | 3.2 | 3.2 |
| benzoxazine | | | | | | | | |
| Poly(S—B—S) | 4 | | | | 6.7 | 6.7 | 6.7 | 6.7 |
| EPDM Trilene 67 | 2.2 | | | | | | | |
| Kraton 1535H | | 6.6 | 6.6 | | | | | |
| Kraton 1536H | | | | | | | 2 | 2 |
| SLK1500 | | 1.9 | | | | | | |
| SLK3000 | | | | | | 1.9 | | |
| MIR5000 | | | 1.9 | | 1.9 | | | |
| Septon 1020 | | | | 6.6 | | | | |
| M1913 | | | | | | | 2 | |
| Septon V9461 | | | | | | | | 2 |
| Properties | | | | | | | | |
| Dk SPC | N/A | N/A | N/A | N/A | 3.3 | 3.41 | 3.47 | 3.37 |
| Df SPC | N/A | N/A | N/A | N/A | 0.0023 | 0.0027 | 0.002 | 0.002 |

TABLE 3

| Example | 9 | 10 | 11 | 12 | 13 | 14 | 15 |
|---|---|---|---|---|---|---|---|
| CCDFB | 0.4 | 0.4 | 0.4 | 0.4 | 0.4 | 0.4 | 0.8 |
| Dicumyl peroxide | | | | | 0.1 | | |
| Saytex 8010 | 8 | 8 | 8 | 8 | 8 | 7.5 | 11.1 |
| Alumina AX-32 | 18.3 | 18.3 | 18.3 | 18.3 | 18.3 | 17.2 | 17.1 |
| Silica SS-15V | 53.4 | 53.4 | 53.3 | 53.3 | 53.4 | 50 | 46.2 |
| KR511 | | | 0.3 | | | | |
| OFS-6518 | | | | 0.3 | | | |
| OFS-6030 | | | | | 0.5 | 0.6 | 0.6 |
| poly(butadiene) | 2 | | | | | | |
| 1,3-phenyl | 1.9 | 1.9 | 1.9 | 1.9 | 1.9 | 2.1 | 2.1 |
| bismaleimide | | | | | | | |
| Bisphenol A | 3.2 | 3.2 | 3.1 | 3.1 | 2.6 | 2.9 | 2.9 |
| benzoxazine | | | | | | | |
| Poly(S—B—S) | 6.7 | 6.7 | 5.6 | 5.6 | 5.7 | 6.4 | 1.2 |
| EPDM Trilene 67 | 2.1 | 6.1 | 4 | 4 | 4.1 | 4.6 | 4.6 |
| Kraton 1536H | 2 | | | | | 2.6 | 2.6 |
| SLK3000 | | 2 | | | | | |
| BMI-689 | 2 | | | | | | |
| BMI-TMH | | | 5 | 5 | 5.1 | 5.7 | 5.8 |
| Septon V9461 | | | | | | | 4.9 |
| Properties | | | | | | | |
| Dk SPC | 3.59 | 3.48 | 3.7 | 3.8 | 3.5 | 3.48 | 3.4 |
| Df SPC | 0.0018 | 0.0018 | 0.0018 | 0.0019 | 0.0016 | 0.0012 | 0.0015 |

TABLE 4

| Example | 16 | 17 | 18 | 19 | 20 | 21 | 22 | 23 | 24 | 25 | 26 | 27 |
|---|---|---|---|---|---|---|---|---|---|---|---|---|
| CCDFB | 0.8 | 0.8 | 0.8 | 0.8 | 0.6 | 0.6 | 0.6 | 0.6 | 0.6 | 0.6 | 0.6 | 0.6 |
| Saytex 8010 | 11.2 | 11.2 | 11.2 | 11.2 | 11.9 | 11.9 | 11.9 | 11.9 | 11.9 | 11.9 | 11.9 | |
| Alumina AX- | 17.1 | 17.1 | 17.1 | 17.1 | 18.3 | 18.3 | 18.3 | 18.3 | 18.3 | 18.3 | 18.3 | |
| 32 | | | | | | | | | | | | |
| SS-15V silica | 46.1 | 46.1 | 46.1 | 46.1 | 49.3 | 49.3 | 49.3 | 49.3 | 49.3 | 49.3 | 49.3 | 63.6 |
| OFS-6030 | 0.6 | 0.6 | 0.6 | 0.6 | 0.5 | 0.5 | 0.5 | 0.5 | 0.5 | 0.5 | 0.5 | 0.5 |
| 1,3-phenyl | | | 2.1 | | 1.7 | | | | | | 1.7 | |
| bismaleimide | | | | | | | | | | | | |
| Bisphenol A | 2.9 | 2.9 | 2.9 | 2.9 | 2.3 | 2.3 | 2.3 | 2.3 | 2.3 | 2.3 | 2.3 | 2.3 |
| benzoxazine | | | | | | | | | | | | |
| Poly(S—B—S) | 4 | 4 | 4 | 4 | 3.2 | 3.2 | 3.2 | 3.2 | 3.2 | | | |
| EPDM | 4.6 | 4.6 | | 4.6 | | | | | | | | |
| Trilene 65 | | | | | | | | | | | | |
| Kraton 1536H | 2.6 | 2.6 | 2.6 | 2.6 | 2.1 | 2.1 | 2.1 | 2.1 | 2.1 | 2.1 | 2.1 | 3.2 |
| BMI689 | | | | | | | | 1.7 | 1.7 | | | |
| BMI-TMH | 5.7 | 5.7 | 5.7 | 5.7 | 4.6 | 6.3 | 6.3 | 4.6 | 4.6 | 6.3 | 4.6 | 8.2 |
| Septon V9461 | 2.4 | 2.4 | 2.4 | 2.4 | 1.9 | 1.9 | 1.9 | 1.9 | 1.9 | 1.9 | 1.9 | |
| MDAB | 2.1 | | | | | | | | | | | |
| MXBI | | 2.1 | | | | | | | | | | |
| PPE/butadiene | | | 4.6 | | | | | | | | | |
| copolymer | | | | | | | | | | | | |
| EH-BMI- | | | | 2.1 | | | | | | | | |
| 2M2E | | | | | | | | | | | | |
| SA-9000 | | | | | 3.7 | 3.7 | | | 3.7 | | 3.2 | |
| JSR HC-21 | | | | | | | 3.7 | 3.7 | | 3.7 | | 5.7 |
| Resin | | | | | | | | | | | | |
| Hydrogenated | | | | | | | | | | 3.2 | | |
| poly(SBS) | | | | | | | | | | | | |
| EPDM | | | | | | | | | | | 3.7 | |
| Trilene 67 | | | | | | | | | | | | |
| SPV-100 | | | | | | | | | | | | 0.6 |
| W-1o | | | | | | | | | | | | 2.9 |
| PQ-60 | | | | | | | | | | | | 12.4 |
| Properties | | | | | | | | | | | | |
| Dk by SPC | 3.515 | 3.615 | 3.745 | 3.565 | 3.535 | 3.555 | 3.565 | 3.565 | 3.52 | 3.51 | 3.505 | 3.27 |
| Df by SPC | 0.0014 | 0.0014 | 0.0016 | 0.0018 | 0.0015 | 0.0017 | 0.0018 | 0.0016 | 0.0016 | 0.0017 | 0.0019 | 0.0017 |

TABLE 5

| Example | 28 | 29 | 30 | 31 | 32 | 33 | 34 | 35 | 36 | 37 | 38 |
|---|---|---|---|---|---|---|---|---|---|---|---|
| alumina AX-32 | 17.1 | 17.1 | 17.1 | 17.1 | 17.1 | 14.9 | | | | | |
| SS-15V silica | 46 | 46 | 46 | 46 | 46 | 44.6 | 51.3 | 52 | 63.5 | 63.5 | 63.5 |
| OFS-6030 | 0.6 | 0.6 | 0.6 | 0.6 | 0.6 | 0.6 | 0.6 | 0.6 | 0.5 | 0.5 | 0.5 |
| CCDFB | 0.8 | 0.8 | 0.8 | 0.8 | 0.8 | 0.8 | 0.8 | 0.8 | 0.6 | 0.6 | 0.6 |
| 1,3-phenyl | 2.1 | 2.1 | 2.1 | 2.1 | 2.1 | 2.1 | | | | | |
| bismaleimide | | | | | | | | | | | |
| Bisphenol A | 2.9 | 2.9 | 2.9 | 2.9 | 2.9 | 2.9 | 3 | 2.9 | 0.2 | 2.3 | 2.3 |
| benzoxazine | | | | | | | | | | | |
| Poly(S—B—S) | 3.9 | 3.9 | 3.9 | 3.9 | 3.9 | 3.9 | | 3.9 | | | |
| EPDM resin, | 4.6 | 4.6 | 4.6 | 4.6 | 4.6 | 4.6 | 4.8 | 2.9 | | | |
| Trilene65 | | | | | | | | | | | |
| Kraton 1536H | 2.6 | 2.6 | 2.6 | 2.6 | 2.6 | 2.6 | 2.7 | 2.6 | 3.2 | 3.2 | 3.2 |
| BMI-TMH | 5.7 | 5.7 | 5.7 | 5.7 | 5.7 | 5.7 | | 7.8 | 8.2 | 8.2 | 8.2 |
| Septon 1020 | 2.4 | 2.4 | 2.4 | 2.4 | 2.4 | 2.4 | 2.5 | 2.4 | | | |
| JSR HC-21 | | | | | | | 12.2 | | 5.7 | 5.7 | 5.7 |
| Resin | | | | | | | | | | | |
| SA-9000 | | | | | | | | 1.7 | | | |
| PQ-60 | | | | | | 11.1 | 18.3 | 18.6 | 7.9 | 13.3 | 7.9 |
| SPV-100 | 11.1 | | | | | | | | | 2.6 | 4 |
| W-1o | | | | | | 3.7 | | 3.7 | 4 | | 4 |
| FP-72TP | | 11.1 | | | | | | | 4 | | |
| BES5-1150 | | | 11.1 | | | | | | | | |
| resorcinol bis | | | | 11.1 | | | 3.7 | | | | |
| (2,6-xylyl | | | | | | | | | | | |
| phosphate) | | | | | | | | | | | |
| phenoxycyclo- | | | | | 11.1 | | | | | | |
| phosphazene | | | | | | | | | | | |
| Properties | | | | | | | | | | | |
| Dk by SPC | N/A | N/A | 3.35 | N/A | N/A | 3.065 | N/A | N/A | N/A | N/A | N/A |
| Df by SPC | N/A | N/A | 0.00158 | N/A | N/A | 0.00164 | N/A | N/A | N/A | N/A | N/A |

As shown in Tables 1-3, curable compositions 5-27 (all of which included both a maleimide-containing compound and a low dielectric loss polymer) surprisingly exhibited superior electrical properties (e.g., relatively low Dk and Df).

### Prophetic Example 39: Preparation and Characterization of Curable Composition 39

To a container are added 0.082 lbs of 1,6-bismaleimide (2,2,4-trimethyl)hexane and 0.224 lbs of toluene. While the solution thus obtained is being stirred, the following chemicals are added sequentially and mixed: 0.057 lbs of a functionalized pyrimidine aryl ether copolymer (JSR HC-21) available from Japanese Synthetic Rubber Corporation, 0.0229 lbs of 2,2′-bis(4-cyanatophenyl)isopropylidene (BA-200) available from Arxada Primaset, 0.032 lbs of Kraton 1536 SEBS poly(styrene-ethylene-butylene-styrene) copolymer, 0.006 lbs of 2,3-dimethyl-2,3-diphenylbutane, 0.005 lbs of methacryloxypropyl trimethoxysilane, 0.635 lbs of a spherical 3 micron solid silica SS-15V available from Sibelco, 0.106 lbs of an organic phosphorous-containing flame retardant diphenylphosphoryl){p-[(diphenylphosphoryl)methyl]phenyl}methane (BES5-1150) available from Regina Electronic Materials, and 0.053 lbs of an allyl-containing phosphazene SPV-100 available from Otsuka Chemical Co. LTD. The BES5-1150 is treated by an Air Classifying Mill to reduce all particles below microns. Finally, 0.055 lbs of toluene is added to the mixture to achieve a specific gravity of about 1.5. The mixture is placed on a high shear mixer and mixed until uniform. The mixture thus obtained is coated onto a release paper using a drawn down wrapped wire rod. The toluene is evaporated from the resulting film by heating the coated release paper at 155° C. for 10 minutes. A free standing film is separated from the release paper and laminated with copper at 450° F. for 2 hours to form a copper clad laminate.

### Prophetic Example 40: Preparation and Characterization of Curable Composition 40

To a container are added first 0.082 lbs of 1,6-bismaleimide (2,2,4-trimethyl)hexane and 0.224 lbs of toluene. While the solution thus obtained is being stirred, the following chemicals are added sequentially and mixed: 0.057 lbs of a functionalized pyrimidine aryl ether copolymer (JSR HC-21) available from Japanese Synthetic Rubber Corporation, 0.0229 lbs of DT-4000 (a multifunctional cyanate ester resin derived from low molecular weight oligomers of dicyclopentadiene and phenols) available from Arxada Primaset, 0.032 lbs of Kraton 1536 SEBS poly(styrene-ethylene-butylene-styrene) copolymer, 0.006 lbs of 2,3-dimethyl-2,3-diphenylbutane, 0.005 lbs of methacryloxypropyl trimethoxysilane, 0.635 lbs of a spherical 3 micron solid silica SS-15V available from Sibelco, 0.106 lbs of an organic phosphorous-containing flame retardant diphenylphosphoryl){p-[(diphenylphosphoryl)methyl]phenyl}methane (BES5-1150) available from Regina Electronic Materials, and 0.053 lbs of an allyl-containing phosphazene SPV-100 available from Otsuka Chemical Co. LTD. Finally, 0.055 lbs of toluene is added to the mixture to achieve a specific gravity of about 1.5. The mixture is placed on a high shear mixer and mixed until uniform. The mixture thus obtained is coated onto a release paper using a drawn down wrapped wire rod. The toluene is evaporated from the resulting film by heating the coated release paper at 155° C. for 10 minutes. A free standing film is separated from the release paper and laminated with copper at 450° F. for 2 hours to form a copper clad laminate.

### Example 41: Preparation and Characterization of Curable Composition 41

To a container were added first 0.082 lbs of 1,6-bismaleimide (2,2,4-trimethyl)hexane and 0.224 lbs of toluene. While the solution thus obtained was being stirred, the following chemicals were added sequentially and mixed: 0.057 lbs of a functionalized pyrimidine aryl ether copolymer (JSR HC-21) available from Japanese Synthetic Rubber Corporation, 0.0229 lbs of bisphenol A benzoxazine (Araldite MT35610) available from Huntsman, 0.032 lbs of Kraton 1536 SEBS poly(styrene-ethylene-butylene-styrene) copolymer, 0.006 lbs of 2,3-dimethyl-2,3-diphenylbutane, 0.005 lbs of methacryloxypropyl trimethoxysilane, 0.635 lbs of a spherical 3 micron solid silica SS-15V from Sibelco, 0.106 lbs of an phosphorous-containing flame retardant diphenylphosphoryl){p-[(diphenylphosphoryl)methyl]phenyl}methane (BES5-1150) available from Regina Electronic Materials, and 0.053 lbs of an allyl-containing phosphazene SPV-100 available from Otsuka Chemical Co. LTD. Finally, 0.055 lbs of toluene was added to the mixture to achieve a specific gravity of about 1.5. The mixture was placed on a high shear mixer and mixed until uniform. The mixture thus formed was coated onto a release paper using a drawn down wrapped wire rod. The toluene was evaporated from the resulting film by heating the coated release paper at 155° C. for 10 minutes. A free standing film was separated from the release paper and laminated with copper at 450° F. for 2 hours to make a copper clad laminate. All samples from the laminate passed an 80 second thermal shock test at 288° C. and showed no signs of blistering. Etched unclad samples passed UL94 V-0 flame testing. Copper peel strength values were 3.48 lbs with ½ oz HS1 VSP copper. An unclad laminate sample had a Dk of 3.25 and Df of 0.00171 by using IPC TM-650 Method 2.5.5.13 Split Post Cavity Measurement.

### Prophetic Example 42: Preparation and Characterization of Curable Composition 42

Curable composition 42 is prepared using the same method described in Example 41 except that the 1,6-bismaleimide (2,2,4-trimethyl)hexane is treated with an air classifying mill to reduce all particles to less than 10 microns.

### Example 43: Preparation and Characterization of Curable Composition 43

To a container were added 0.224 lbs of toluene and 0.046 lbs of an EPDM rubber poly(ethylene-propylene-diene), which was a dicyclopentadiene cured elastomer known as Trilene-65, 0.026 lbs of Kraton 1536 SEBS, 0.0285 lbs of bisphenol A benzoxazine, 0.008 lbs of 2,3-dimethyl-2,3-diphenylbutane, 0.006 lbs of methacryloxypropyl trimethoxysilane, 0.024 lbs of NP2-R (a para methyl styrene functionalized copolymer of styrene and butadiene) available from Kraton, 0.039 lbs of a poly(styrene-butadiene) copolymer having a majority component of butadiene available from Nisso Chemical as SBS-A, 0.460 lbs of spherical 3 micron solid silica SS-15V, 0.171 lbs of spherical alumina AX-32 available from Sibelco, and 0.171 lbs of a brominated flame retardant Saytex 8010, 0.021 lbs of 1,3-phenylene bismaleimide, 0.057 lbs of 1,6-bismaleimide (2,2,4-trimethyl)hexane, and 0.055 lbs of toluene. The mixture was coated onto a release paper using a drawn down wrapped wire rod. The toluene was evaporated from the resulting film by heating the coated release paper at 155° C. for 10 minutes. A free standing film was separated from the release paper and laminated with copper at 450° F. for 2 hours to make a copper clad laminate.

All laminate samples passed an 80 second thermal shock test at 288° C. and showed no signs of blistering. Etched unclad samples passed UL-94 V-0 flame testing. Copper peel strength values were 3.10 lbs with 0.5 oz HS1-M2-VSP. An unclad laminate sample had a Dk of 3.32 and DF of 0.0018 by using IPC TM-650 Method 2.5.5.13 Split Post Cavity Measurement. The tensile modulus of etched laminate samples was 417 MPa. The coefficient of thermal expansion from an etched laminate sample from room temperature to 260° C. was 16 ppm/° C. The non-reinforced resin composition achieved a dimensional movement (shrinkage) of 0.55 mils/in after pressing in the machine direction and 0.6 mils/inch (IPC-650 2.2.4 [TS]).

### Prophetic Example 44: Preparation and Characterization of Curable Composition 44

Curable composition 44 is prepared using the same method described in Example 43 except that the 1,3-phenylene bismaleimide and 1,6-bismaleimide (2,2,4-trimethyl)hexane are treated with an air classifying mill and the particle sizes of both materials are reduced to less than 10 microns.

### Example 45: Preparation and Characterization of Curable Composition 45

To a container were added 0.082 lbs of 1,6-bismaleimide (2,2,4-trimethyl)hexane and 0.224 lbs of toluene. While the solution thus obtained was being stirred, the following chemicals were added sequentially and mixed: 0.057 lbs of a functionalized pyrimidine aryl ether copolymer (JSR HC-21) available from Japanese Synthetic Rubber Corporation, 0.0229 lbs of bisphenol A benzoxazine (Araldite MT35610) available from Huntsman, 0.032 lbs of Kraton 1536 SEBS poly(styrene-ethylene-butylene-styrene) copolymer, 0.006 lbs of 2,3-dimethyl-2,3-diphenylbutane, 0.005 lbs of methacryloxypropyl trimethoxysilane, 0.243 lbs of a spherical 3 micron solid silica SS-15V from Sibelco, 0.095 lbs of HS-200 hollow silica (density 0.5-0.6 g/cm3, d50=2 microns, DK=1.5-1.6, DF=0.001) available from AGC Inc., 0.106 lbs of an organic phosphorous-containing flame retardant diphenylphosphoryl){p-[(diphenylphosphoryl)methyl]-phenyl}methane (BES5-1150) available from Regina Electronic Materials, and 0.053 lbs of an allyl-containing phosphazene SPV-100 available from Otsuka Chemical Co. LTD. Finally, 0.055 lbs of toluene was added to the mixture to achieve a specific gravity of about 1.5. The resin composition was then coated onto a polymer release coated paper using a wire-wound wrapped iron Mayer wet film applicator rod to a consistent thickness. The resin composition on the paper carrier was then heated at 150° C. for ten minutes or until all toluene was evaporated. The consolidated resin composition was then separated from the release coated paper to yield a free standing resin film. The free standing resin film was then laminated between two pieces of 0.5 oz HS1 VSP copper to yield a double sided copper clad laminate. Portions of the laminate sample were etched to remove the copper for dielectric property measurements. Measurements by a Dielectric Sheet Tester (Damaskos, Inc., Model 03 Thin Sheet Dielectric Tester) at 6.5 GHz showed a DK of 2.18 and a DF of 0.00132.

### Example 46: Preparation and Characterization of Curable Composition 46

To a container were added 0.122 lbs of 1,6-bismaleimide (2,2,4-trimethyl)hexane and 0.1 lbs of toluene. While the solution thus obtained was being stirred, the following chemicals were added sequentially and mixed: 0.212 lbs of a functionalized pyrimidine aryl ether copolymer (JSR HC-30) available from Japanese Synthetic Rubber Corporation, 0.034 lbs of bisphenol A benzoxazine (Araldite MT35610) available from Huntsman, 0.048 lbs of Kraton 1536 SEBS poly(styrene-ethylene-butylene-styrene) copolymer, 0.009 lbs of 2,3-dimethyl-2,3-diphenylbutane, 0.007 lbs of methacryloxypropyl trimethoxysilane, 0.181 lbs of HS-200 hollow silica available from AGC Inc., 0.151 lbs of an organic phosphorous-containing flame retardant diphenylphosphoryl){p-[(diphenylphosphoryl)methyl]phenyl}methane (BES5-1150) available from Regina Electronic Materials, and 0.0789 lbs of an allyl-containing phosphazene SPV-100 available from Otsuka Chemical Co. LTD. Finally, 0.05 lbs of toluene was added to the mixture to achieve a specific gravity of about 1.5. The resin composition was then coated onto a polymer release coated paper using a wire-wound wrapped iron Mayer wet film applicator rod to a consistent thickness. The resin composition on the paper carrier was then heated at 150° C. for ten minutes or until all toluene was evaporated. The consolidated resin composition was then separated from the release coated paper to yield a free standing resin film. The free standing resin film was then laminated between two pieces of 0.5 oz HS1 VSP copper to yield a double sided copper clad laminate. Portions of the laminate sample were etched to remove the copper for dielectric property measurements. An unclad laminate sample had a Dk of 2.0 and Df of 0.00188 at 10 GHz by using IPC TM-650 Method 2.5.5.13 Split Post Cavity Measurement. The coefficient of thermal expansion from room temperature to 260° C. was 41.5 ppm/° C.

### Example 47: Preparation of Laminate Using the Film Obtained from Example 41

The free standing dielectric film obtained from Example 41 was used together with a microthin copper attached to a copper carrier. Specifically, a 2.5 mil thick free standing dielectric film obtained from Example 41 was laminated to an FR4 epoxy substrate with exposed bare dielectric. Subsequently, a microthin 3.0 micron copper (Rz=0.9 microns) bonded to a 0.5 oz copper carrier (Doublethin™ NN, available from Circuit Foil) was laminated to the top surface of the dielectric film. The lamination conditions were 450° F. for 2 hours at 100 psi. The carrier copper was subsequently removed. The microthin copper was plated up to 18 microns using electrolytic plating batch. The subsequent peel strength measured 2 lbs/in. A similar experiment was carried out with Doublethin DTH ANP 3 micron copper (Rz=1.2 microns) and a peel strength of 3 lbs/inch was measured.

### Example 48: Preparation of Resin Coated Copper Using the Curable Composition Obtained From Example 41

A five hundred yard roll of a microthin 1.5 micron copper bonded to a 0.5 oz copper carrier (Doublethin™ NN, available from Circuit Foil) was coated with the resin composition prepared in Example 41. The resin composition was coated at a coating rate of 8 ft/min. onto the top surface of the microthin copper by using a wire-wound wrapped iron Mayer wet film applicator rod to a consistent thickness and by using a coating tower. The 30 foot drying stage of the coating tower had a peak temperature of 310° F.

This example demonstrates the preparation of a resin coated copper. In some embodiments, the copper can be a microthin copper bonded to a ⅜^{th} oz, 0.5, oz or 1.0 oz copper carrier. In some embodiments, the coating composition of Example 41 can be simply coated onto the treated side (or the nodule side) of a conventional 0.5 or 1.0 oz copper such as Circuit Foils BF-ANP, BF-NN, BF-HFZ, HFZ, HFZ-LP, and reverse treated copper HFZ-B.

### Example 49: Preparation of Resin Coated Aluminum Using the Curable Composition Obtained From Example 41

A five hundred yard roll of an aluminum foil treated on one face with a 10 nanometer thick layer of palladium was coated with the resin composition prepared in Example 41. The resin composition was coated at a coating rate of 8 ft/min. onto the palladium face using a wire-wound wrapped iron Mayer wet film applicator rod to a consistent thickness and by using a coating tower. The 30 foot drying stage of the coating tower had a peak temperature of 310° F.

### Example 50: Preparation of Multilayer Laminate Using the Curable Composition Obtained From Example 41

A thin core laminate was prepared using two total plies of films prepared from Curable Composition 41 and a single ply of bare 2116 E-glass. The thin core laminate included a single sheet of 2116 E-glass between two plies of 0.0025 inch thick films prepared from Curable Composition 41, with a single ply on the bottom and a single ply on top of the 2216 E-glass. This laminate (which included, from top to bottom, Curable Composition 41-2216 E-glass—Curable Composition 41) was vacuum pressed between two layers of copper at 450° C. for 120 minutes at 250 psi. Copper peel strength values were 2.15 lb/in using ½ oz HS1-M2-VSP copper. An unclad laminate sample had a Dk of 3.44 and Df of 0.00197 using IPC TM-650 Method 2.5.5.13 Split Post Cavity Measurement. A CTE in the X-Y direction was measured by TMA to be 11 ppm/° C. All samples passed an 80 second thermal shock test at 288° C. and showed no signs of blistering.

A thin core laminate to achieve extremely low CTE was prepared using a 0.0025-inch-thick film prepared from Curable Composition 41, a 0.0012-inch-thick film prepared from Curable Composition 41, and 2113 SI-glass. Specifically, the following laminate was prepared by pressing between two layers of copper (from top to bottom): a 0.0025 inch thick film prepared from Curable Composition 41, 2113 SI-glass, a 0.0025-inch-thick film prepared from Curable composition 41, 2113 SI-glass, and a 0.0025-inch-thick film prepared from Curable composition 41. This laminate was formed by vacuum pressing at 450° C. for 120 minutes at 250 psi.

In another example, the following laminated was prepared by pressing between two layers of copper (from top to bottom): a 0.0025 inch thick film prepared from Curable Composition 41, 2113 SI-glass, a 0.0012-inch-thick film prepared from Curable composition 41, 2113 SI-glass, and a 0.0025-inch-thick film prepared from Curable composition 41. This laminate was formed by vacuum pressing at 450° C. for 120 minutes at 250 psi.

Examples 51-73 in Tables 6 and 7 were made using the same procedures and characterization methods as the Examples 1-50. The amount of each component in Tables 6 and 7 is in part by weight.

TABLE 6

| Example | 51 | 52 | 53 | 54 | 55 | 56 | 57 | 58 | 59 | 60 | 61 |
|---|---|---|---|---|---|---|---|---|---|---|---|
| 2,3-dimethyl-2,3-diphenylbutane | 0.006 | 0.009 | 0.009 | 0.006 | 0.009 | 0.010 | 0.008 | 0.010 | 0.014 | 0.015 | 0.012 |
| Megasil SS-15V solid silica | 0.635 | 0.615 | 0.615 | 0.635 | 0.615 | 0.385 | 0.545 | | | | 0.067 |
| EQ1010 solid Silica | | | | | | | | 0.216 | | | |
| OFS-6030 | 0.005 | 0.007 | 0.007 | 0.005 | 0.007 | 0.008 | 0.006 | | | 0.005 | 0.009 |
| Methacryloxypropylsilane | | | | | | | | | | | |
| Hollow Silica (HS-200, Untreated) | | | | | | 0.151 | 0.065 | | | | |
| Hollow Silica (HS-200-MT, | | | | | | | | 0.197 | | | |
| Methacrylate Treated) | | | | | | | | | | | |
| Hollow Silica (HS-200-TM, TM | | | | | | | | | 0.176 | 0.199 | 0.269 |
| Treated) | | | | | | | | | | | |
| Bisphenol A benzoxazine | 0.023 | 0.027 | 0.027 | 0.023 | 0.027 | 0.018 | 0.015 | 0.038 | 0.050 | 0.057 | |
| Vector4411 Poly (SIS) Dexco | | 0.015 | 0.015 | | 0.015 | | | | | | |
| Polymers | | | | | | | | | | | |
| Kraton 1536H SEBS | | | | | | 0.010 | 0.009 | 0.053 | | | |
| Kraton 1648 SEBS | 0.032 | | | 0.032 | | | | | 0.071 | | |
| Kraton D1623 SEBS | | 0.02 | | | | | | | | 0.04 | 0.052 |
| BMI-TMH | 0.082 | 0.071 | 0.057 | 0.082 | 0.057 | 0.130 | 0.109 | 0.134 | 0.179 | | |
| JSR HC-21 Resin | | | | | | 0.036 | 0.030 | | | | |
| JSR HC-30 Resin | 0.057 | 0.058 | 0.058 | 0.057 | 0.058 | | | 0.093 | 0.125 | 0.142 | 0.104 |
| SPV-100 Otsuka Chemicals tris(2- | 0.053 | 0.069 | 0.103 | | | 0.084 | 0.071 | 0.087 | 0.116 | 0.132 | 0.108 |
| allylphenoxy)triphenoxy | | | | | | | | | | | |
| cyclotriphosphazene | | | | | | | | | | | |
| Regina BES5-1150 p-xylylene-bis- | 0.106 | 0.108 | 0.108 | 0.106 | 0.108 | 0.168 | 0.141 | 0.173 | 0.123 | 0.139 | 0.144 |
| diphenylphosphine oxide | | | | | | | | | | | |
| Fushimi FP-300 cyanophenoxy | | | | 0.053 | 0.103 | | | | | | |
| phenoxy(cyclophosphazene) | | | | | | | | | | | |
| Regina BVPE bis(4- | | | | | | | | | 0.146 | 0.073 | |
| vinylhenyl)ethane | | | | | | | | | | | |
| Shikoku kasei LDAIC (allyl modified | | | | | | | | | | | 0.026 |
| triazine) | | | | | | | | | | | |
| Denka LDM-03 divinylbenzene- | | | | | | | | | | | 0.095 |
| styrene-ethylene terpolymer | | | | | | | | | | | |
| Huntsman LME (modified | | | | | | | | | | 0.2 | 0.114 |
| divinylbenzene) | | | | | | | | | | | |
| Characterization | | | | | | | | | | | |
| Dk by SPC | 3.23 | 3.18 | | | | 2.36 | 2.75 | 2.06 | 2.15 | | |
| Df by SPC | 0.00188 | 0.0023 | | | | 0.0017 | 0.00147 | 0.00148 | 0.0017 | | |
| Copper Peels, ½ oz HS1-VSP | 2.32 | 1.7 | | | | | | | | | |
| Solder Float | Pass | Pass | | | | | Pass | | | | |
| Passed VO | Pass | Pass | | | | | | | | | |
| CTE (40-260) | 30 | | | | | 30.8 | 26.6 | 37.8 | | | 34 |
| Resin Flow (%) | 39% | | | | | | | | 80.7 | 95 | |
| Min. Melt Viscosity (Poise) | | | | | | | | | 11000 | | |

TABLE 7

| Example | 62 | 63 | 64 | 65 | 66 | 67 | 68 | 69 | 70 | 71 | 72 | 73 |
|---|---|---|---|---|---|---|---|---|---|---|---|---|
| 2,3-dimethyl-2,3- | 0.006 | 0.0134 | 0.0134 | 0.0134 | 0.0138 | 0.0138 | 0.0138 | 0.0138 | 0.0137 | 0.0137 | 0.0137 | 0.0137 |
| diphenylbutane | | | | | | | | | | | | |
| Megasil SS-15V solid | 0.075 | | | | | | | | | | | |
| silica | | | | | | | | | | | | |
| OFS-6030 Metha- | 0.005 | 0.0043 | 0.0043 | 0.0043 | 0.0044 | 0.0044 | 0.0044 | 0.0044 | 0.0044 | 0.0044 | 0.0044 | 0.0044 |
| cryloxypropylsilane | | | | | | | | | | | | |
| Hollow Silica (HS-200- | | | | | 0.1901 | 0.1901 | 0.1901 | 0.1901 | | | | |
| MT, Methacrylate | | | | | | | | | | | | |
| Treated) | | | | | | | | | | | | |
| Hollow Silica (HS-200- | 0.208 | 0.1956 | 0.1956 | 0.1956 | | | | | 0.1966 | 0.1966 | 0.1966 | 0.1966 |
| TM, TM Treated) | | | | | | | | | | | | |
| P-d Benzoxazine | | | | | | 0.1138 | | | | | | |
| (Shikoku bisphenyl F | | | | | | | | | | | | |
| based benzoxazine) | | | | | | | | | | | | |
| Bisphenol A benzoxazine | 0.083 | 0.1108 | 0.1108 | 0.1108 | 0.1138 | | 0.0586 | | 0.0581 | 0.0581 | 0.1129 | 0.0581 |
| Vector4411 Poly (SIS) | | | | 0.0269 | 0.0506 | 0.0506 | 0.1241 | 0.1241 | 0.0502 | 0.0502 | 0.025 | 0.0502 |
| Dexco Polymers | | | | | | | | | | | | |
| Kraton D1623 SEBS | 0.05 | 0.0717 | 0.0717 | 0.0717 | 0.0735 | 0.0735 | | | 0.073 | 0.073 | 0.073 | 0.073 |
| BMI-TMH | 0.039 | | | | | | | 0.0897 | | 0.024 | | |
| JSR HC-30 Resin | | 0.1403 | 0.1403 | 0.1403 | 0.144 | 0.144 | 0.144 | 0.144 | 0.1966 | 0.1736 | 0.1428 | 0.1736 |
| SPV-100 Otsuka | 0.104 | 0.2248 | 0.2248 | 0.2248 | 0.2307 | 0.2307 | 0.2307 | 0.2307 | 0.2289 | 0.2289 | 0.2289 | 0.2528 |
| Chemicals tris(2- | | | | | | | | | | | | |
| allylphenoxy)tri- | | | | | | | | | | | | |
| phenoxy cyclo- | | | | | | | | | | | | |
| triphosphazene | | | | | | | | | | | | |
| Regina BES5-1150 p- | 0.201 | 0.1898 | 0.1898 | 0.1898 | 0.1792 | 0.1792 | 0.1792 | 0.1792 | 0.1777 | 0.1777 | 0.1777 | 0.1777 |
| xylylene-bis- | | | | | | | | | | | | |
| diphenylphosphine oxide | | | | | | | | | | | | |
| Regina BVPE bis(4- | 0.025 | 0.0493 | | | | | | | | | | |
| vinylhenyl)ethane | | | | | | | | | | | | |
| Shikoku kasei LDAIC | | | 0.0493 | 0.0224 | | | | 0.0241 | | | | |
| (allyl modified triazine) | | | | | | | | | | | | |
| Denka LDM-03 | 0.101 | | | | | | | | | | | |
| divinylbenzene-styrene- | | | | | | | | | | | | |
| ethylene terpolymer | | | | | | | | | | | | |
| DIC NEX9470S | | | | | | | | | | | 0.025 | |
| alkylmodified | | | | | | | | | | | | |
| bismaleimide | | | | | | | | | | | | |
| Huntsman LME | | | | | | | 0.0552 | | | | | |
| (modified | | | | | | | | | | | | |
| divinylbenzene) | | | | | | | | | | | | |
| Characterization | | | | | | | | | | | | |
| Dk by SPC | | 2.21 | 2.14 | 2.16 | 2.13 | 2.16 | 2.20 | 2.24 | 2.2 | 2.2 | 2.1 | 2.2 |
| Df by SPC | | 0.0024 | 0.0023 | 0.0025 | 0.0026 | 0.0026 | 0.0021 | 0.0019 | 0.0019 | 0.00194 | 0.0022 | 0.002 |
| Copper Peels, ½ oz | | 1.5 | 1.5 | 1.54 | 1.69 | | | | 1.99 | | 1.5 | |
| HS1-VSP | | | | | | | | | | | | |
| Solder Float | | Pass | Pass | | Pass | | | Pass | | | | |
| Passed VO | | | | | | | | | | | | |
| CTE (40-260) | 64 | 41 | 33 | 41 | 31 | 26 | 39 | | 48 | | 43 | 41 |
| Resin Flow (%) | 83 | 92 | 90 | 79 | 81 | 89 | 47 | 68 | 88 | 77 | | |
| Min. Melt Viscosity | | 3932 | | | 8611 | | | | | | 9787 | |
| (Poise) | | | | | | | | | | | | |

Examples 56-73 are embodiments that show the use of hollow silica. Examples 56-58 demonstrate that a dielectric constant from 2.1 to 2.8 can be readily achieved based on the concentration of hollow silica. Examples 59-73 show that a good balance of other properties can be achieved by varying the total build up film composition. A balance of high flow, low GTE, low Dk, and peel strength is desirable.

Other embodiments are within the scope of the following claims.

What is claimed is:

1. A curable composition, comprising:
at least one benzoxazine compound;
at least one low dielectric loss polymer or a hydrogenated derivative thereof, the at least one low dielectric loss polymer comprising a poly(phenylene ether), or a copolymer comprising a styrene monomer unit, an ethylene monomer unit, a propylene monomer unit, a butylene monomer unit, a butadiene monomer unit, an isoprene monomer unit, a divinylbenzene monomer unit, a pyrimidine monomer unit, or a pyridazine monomer unit;
at least one filler; and
at least one radical initiator.

2. The composition of claim 1, wherein the benzoxazine compound is a bifunctional benzoxazine.

3. The composition of claim 1, wherein the benzoxazine compound is a compound of the following structure: 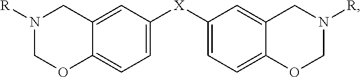
wherein X is an alkylene group, an optionally substituted arylene group, a heteroatom, or a combination of aromatic and aliphatic groups; and R is optionally substituted alkyl, optionally substituted cycloalkyl, allyl, optionally substituted C₆₋₂₂ aryl, optionally substituted 5-22 membered heteroaryl, or an oligomer.

4. The composition of claim 3, wherein X is phenylene, naphthylene, biphenylene, isopropylidene, methylene, a linear C₂₋₂₀ alkylene group, or a heteroatom.

5. The composition of claim 3, wherein X is isopropylidene or methylene.

6. The composition of claim 3, wherein X is oxygen.

7. The composition of claim 3, wherein R is allyl, phenyl, naphthyl, biphenyl, benzyl, or alkyl-substituted phenyl.

8. The composition of claim 3, wherein the benzoxazine compound is a compound of one of the following structures:

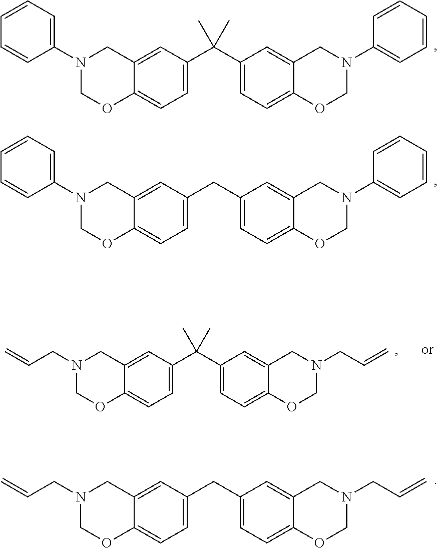

9. The composition of claim 1, wherein the benzoxazine compound is a compound of the following structure:

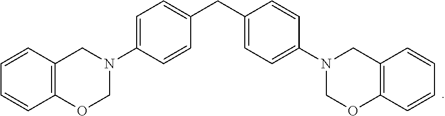 [SMILES: c1ccc2c(c1)CN(c1ccc(Cc3ccc(N4COc5ccccc5C4)cc3)cc1)CO2]

10. The composition of claim 1, wherein the benzoxazine compound is a monofunctional benzoxazine.

11. The composition of claim 1, wherein the benzoxazine compound is a compound of one of the following structures:

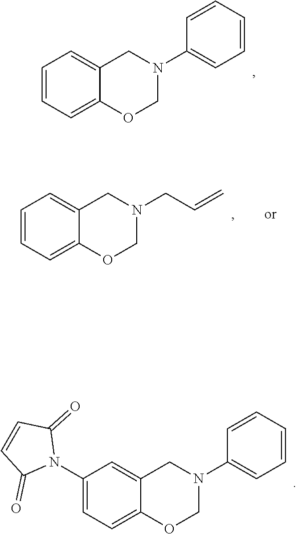 [SMILES: c1ccc(N2COc3ccccc3C2)cc1 ; C=CCN1COc2ccccc2C1 ; O=C1C=CC(=O)N1c1ccc2c(c1)CN(c1ccccc1)CO2]

12. The composition of claim 1, wherein the at least one benzoxazine compound is present in an amount of from about 1 wt % to about 50 wt % of the solid content of the composition.

13. The composition of claim 1, wherein the at least one low dielectric loss polymer comprises a poly(arylene ether) polymer comprising a monomer unit containing a pyrimidine, pyrazine, or pyridazine group.

14. The composition of claim 13, wherein the at least one low dielectric loss polymer comprises a poly(arylene ether) polymer comprising a monomer unit of one of formulas (4)-(8): 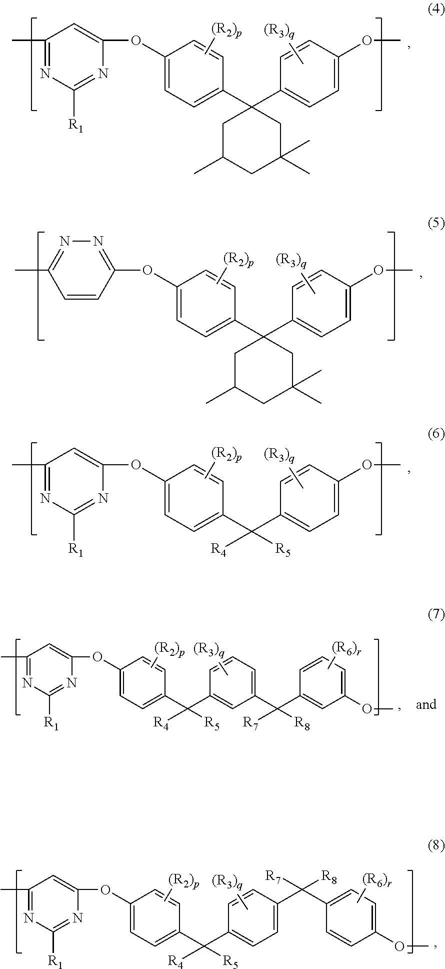
in which R₁ is H, C₁-C₁₀ alkyl, or aryl; each of R₂, R₃, and R₆, independently, is C₁-C₁₀ alkyl; each of R₄, R₅, R₇, and R₈ is H or C₁-C₁₀ alkyl; p is an integer from 0 to 4; q is an integer from 0 to 4; and r is an integer from 0 to 4.

15. The composition of claim 1, wherein the at least one low dielectric loss polymer comprises a poly(phenylene ether) of formula (V): 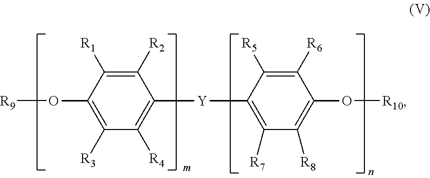
in which
each of m and n, independently, is an integer from 1 to 100;
each of R₁, R₂, R₃, R₄, R₅, R₆, R₇, and R₈, independently, is hydrogen or C₁-C₁₂ alkyl;
each of R₉ and R₁₀, independently, is an end group containing a carbon-carbon double bond; and
Y is a single bond, —C(O)—, —C(S)—, —S(O)—, —S(O)₂—, —C(RR′)—, or 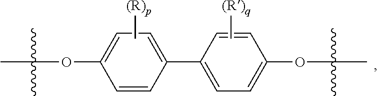
in which each of R and R′, independently, is hydrogen or C₁-C₁₂ alkyl, p is an integer from 0 to 4, and q is an integer from 0 to 4.

16. The composition of claim 1, wherein the at least one low dielectric loss polymer comprises a polyethylene, a polypropylene, a polybutadiene, a polystyrene, a poly(styrene-co-butadiene) copolymer, a polydivinylbenzene copolymer, a poly(styrene-ethylene-butylene-styrene) copolymer, a poly(styrene-ethylene-propylene-styrene) copolymer, a poly(styrene-ethylene-(ethylene-propylene)-styrene) copolymer, a poly(styrene-butadiene-styrene) copolymer, a poly(butadiene-styrene-butadiene) copolymer, a poly(styrene-isoprene-styrene) copolymer, a poly(styrene-propylene-styrene) copolymer, and a poly(ethylene-propylene-diene) copolymer.

17. The composition of claim 1, wherein the at least one low dielectric loss polymer is present in an amount of from about 1 wt % to about 30 wt % of the solid content of the composition.

18. The composition of claim 1, wherein the at least one filler comprises silica, alumina, quartz, titanium dioxide, boron nitride, barium titanate, barium strontium titanate, or polymer particles.

19. The composition of claim 1, wherein the at least one filler comprises hollow particles.

20. The composition of claim 19, wherein the hollow particles comprise hollow silica particles or hollow polymer particles.

21. The composition of claim 1, wherein the particles have an average diameter from about 0.5 μm to about 10 μm.

22. The composition of claim 1, wherein the at least one filler is present in an amount of from about 10 wt % to about 85 wt % of the solid content of the composition.

23. The composition of claim 1, wherein the at least one radical initiator comprises a peroxide, a hydrocarbon, or an azo compound.

24. The composition of claim 23, wherein the at least one radical initiator comprises 2,2′-azobis(2,4,4-trimethylpentane), di-(tert-butylperoxyisopropyl)benzene, bis(1-methyl-1-phenylethyl) peroxide, 2,3-dimethyl 2,3-diphenyl butane, or dicumyl peroxide.

25. The composition of claim 1, wherein the at least one radical initiator is present in an amount of from about 0.1 wt % to about 10 wt % of the solid content of the composition.

26. The composition of claim 1, further comprising at least one organic solvent.

27. The composition of claim 26, wherein the at least one organic solvent comprises tetrahydrofuran, acetonitrile, dimethylformamide, N-methylpyrrolidone, dimethylacetamide, acetone, 2-heptanone, methyl ethyl ketone, methyl isobutyl ketone, methyl n-amyl ketone, methyl isoamyl ketone, cyclopentanone, cyclohexanone, benzene, anisole, toluene, 1,3,5-trimethylbenzene, xylene, propylene glycol monomethyl ether, propylene glycol monomethyl ether acetate, or a combination thereof.

28. The composition of claim 26, wherein the at least one organic solvent is present in an amount of from about 1 wt % to about 50 wt % of the total weight of the composition.

29. The composition of claim 1, further comprising at least one coupling agent.

30. The composition of claim 29, wherein the at least one coupling agent comprises a silane, a siloxane, a polymethylsilsesquioxane, a titanate, or a zirconate.

31. The composition of claim 30, wherein the at least one coupling agent comprises diethoxymethylvinylsilane, trimethoxy(7-octen-1-yl)silane, allyltrimethoxysilane, methacryloxypropyltrimethoxysilane, vinyltrimethoxysilane, triethoxyvinylsilane, hydrolyzed vinylbenzylaminoethylaminopropyltrimethoxy silane, phenyltrimethoxysilane, (p-methylphenyl)trimethoxysilane, p-styryltrimethoxysilane, 3-isocyanatepropyltriethoxysilane, 3-methacryloxypropyl trimethoxysilane, amino ethyl amino trimethoxy silane, amino ethyl amino propyl trimethoxy silane tridecafluoro-1,1,2,2-tetrahydrooctyl(triethoxy), tetra(2,2-diallyloxymethyl-1-butyl)bis(ditridecylphosphite)-titanate, or tetra(2,2-diallyloxymethyl-1-butyl)bis(ditridecylphosphite)zirconate.

32. The composition of claim 29, wherein the at least one coupling agent is present in an amount of from about 0.1 wt % to about 10 wt % of the solid content of the composition.

33. The composition of claim 1, further comprising a flame retardant.

34. The composition of claim 33, wherein the flame retardant comprises 1,1′-(ethane-1,2-diyl)bis(pentabromobenzene), N,N-ethylene-bis(tetrabromophthalimide), aluminum diethylphosphinate, p-xylylene-bis-diphenylphosphine oxide, (2,5-diallyloxyphenyl)diphenylphosphine oxide, hexaphenoxy cyclotriphosphazene, tris(2-allylphenoxy)triphenoxy cyclotriphosphazene, resorcinol bis(di-2,6-dimethylphenyl phosphate), an allyl-containing phosphazene, or a vinyl containing phosphazene.

35. The composition of claim 33, wherein the composition comprises first and second flame retardants, the first flame retardant is insoluble in an organic solvent, and the second flame retardant is soluble in an organic solvent.

36. The composition of claim 35, wherein the weight ratio of the first flame retardant to the second flame retardant is from about 0.5:1 to about 3:1.

37. The composition of claim 33, wherein the flame retardant is present in an amount of from about 1 wt % to about 45 wt % of the solid content of the composition.

38. The composition of claim 1, further comprising at least one cross-linking agent.

39. The composition of claim 38, wherein the at least one cross-linking agent comprises 1,2-bis(4-vinylphenyl)ethane, triallyl cyanurate, triallyl isocyanurate, trimethylallyl isocyanurate, an alkyl functionalized allylsubstituted triazine, a polybutadiene, a poly(butadiene-co-styrene) copolymer, divinylbenzene, a styrene-divinylbenzene copolymer, an ethylene-styrene-divinylbenzene terpolymer, a di(meth)acrylate, bisphenol A diallyl ether, acenapthylene, a cyanate ester, a benzoxazine, or a bismaleimide.

40. The composition of claim 38, wherein the at least one cross-linking agent is present in an amount of from about 1 wt % to about 20 wt % of the solid content of the composition.

41. A film prepared from the composition of claim 1.

42. The film of claim 41, wherein the film is a free standing film.

43. The film of claim 41, wherein the film has a tensile modulus of at most about 10000 MPa.

44. The film of claim 41, wherein the film has a dissipation factor of at most about 0.004.

45. An article, comprising:
a carrier; and
at least one layer supported by the carrier, the at least one layer comprising the film of claim 41.

46. An article comprising:
a fiberglass based substrate core, a glass core with or without metallized through vias, or a TSV; and
from 1 to 10 build up film layers that are laminated on top, bottom or both sides of the substrate core, glass core, or TSV, together with copper metallization such that circuitry are created that is flame retardant and passes UL VO.

47. The article of claim 45, wherein the carrier comprises a metal foil, paper, or a polymer.

48. The article of claim 47, wherein the metal foil is a stainless steel foil, a copper foil, an aluminum foil, or an aluminum foil treated with palladium.

49. A laminate, comprising:
first and second films or a plurality of films; and
a woven or non-woven substrate between the first and second films;
wherein at least one of the first and second films is the film of claim 41.

50. The laminate of claim 49, further comprising a metal foil on a surface of the first or second film.

51. The laminate of claim 50, further comprising first and second metal foils;
wherein the first film is between the first metal foil and the substrate and the second film is between the second metal foil and the substrate.

52. The laminate of claim 49, wherein the substrate comprises a glass cloth.

53. A circuit board for use in an electronic product, comprising the laminate of claim 49.

54. A curable composition, comprising:
at least one benzoxazine compound;
at least one filler;
at least one radical initiator; and
at least one organic solvent.

55. A curable composition, comprising:
at least one maleimide-containing compound comprising a bismaleimide compound or a polymaleimide;
at least one low dielectric loss polymer or a hydrogenated derivative thereof, the at least one low dielectric loss polymer comprising a poly(phenylene ether), or a copolymer comprising a styrene monomer unit, an ethylene monomer unit, a propylene monomer unit, a butylene monomer unit, a butadiene monomer unit, an isoprene monomer unit, a divinylbenzene monomer unit, a pyrimidine monomer unit, or a pyridazine monomer unit;
at least one filler; and
at least one radical initiator.

56. The composition of claim 55, wherein the bismaleimide compound comprises at least one C₄-C₄₀ alkyl group, at least one C₄-C₄₀ alkylene group, at least one aryl group, or at least one heteroaryl group.

57. The composition of claim 55, wherein the bismaleimide compound is 1,6-bis(maleimido)hexane, 1,10-bis(maleimide)decane, 1,3-phenylene bismaleimide, 3,3′-dimethyl-5,5′-diethyl-4,4′-diphenylmethane bismaleimide, m-xylenebismaleimide, N,N′-bismaleimido-4,4′-diphenylmethane, 1,6-bismaleimido(2,2,4-trimethyl)hexane, 4-methyl-1,3-phenylene bismaleimide, 1,3-bis(3-maleimidophenoxy)benzene, 1,3-bis(4-maleimidophenoxy)benzene, 1,3-bis(citraconimidomethyl)benzene, bisphenol A diphenylether bismaleimide, or 2,2′-bis-[4-(4-maleimidophenoxy)phenyl]propane.

58. The composition of claim 55, wherein the bismaleimide compound is a compound of formula (1), (2), or (3): 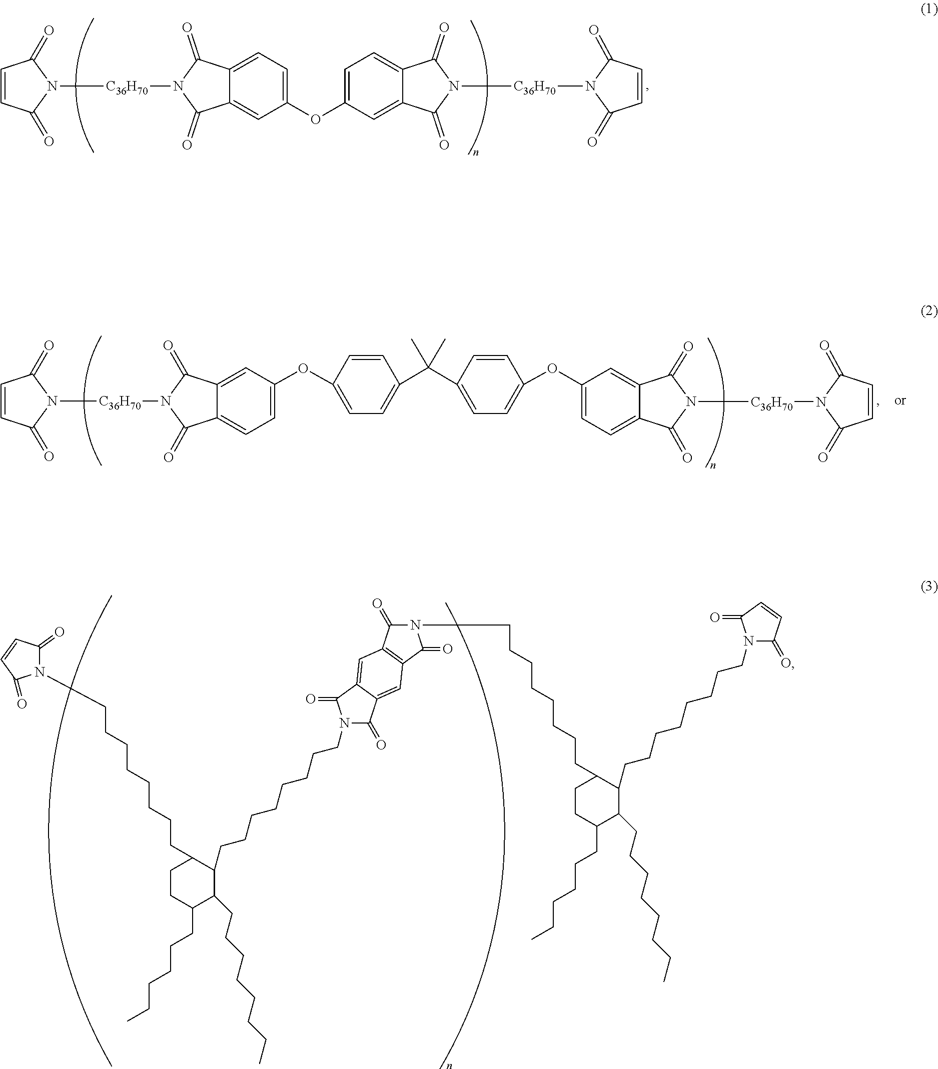
in which n is an integer from 1 to 10, or n is an integer with an average value of about 1.3, or n is an integer with an average value of about 3.1.

59. The composition of claim 55, wherein the at least one maleimide-containing compound is present in an amount of from about 1 wt % to about 50 wt % of the solid content of the composition.

60. The composition of claim 55, wherein the at least one low dielectric loss polymer comprises a poly(arylene ether) polymer comprising a monomer unit containing a pyrimidine, pyrazine, or pyridazine group.

61. The composition of claim 60, wherein the at least one low dielectric loss polymer comprises a poly(arylene ether) polymer comprising a monomer unit of one of formulas (4)-(8): 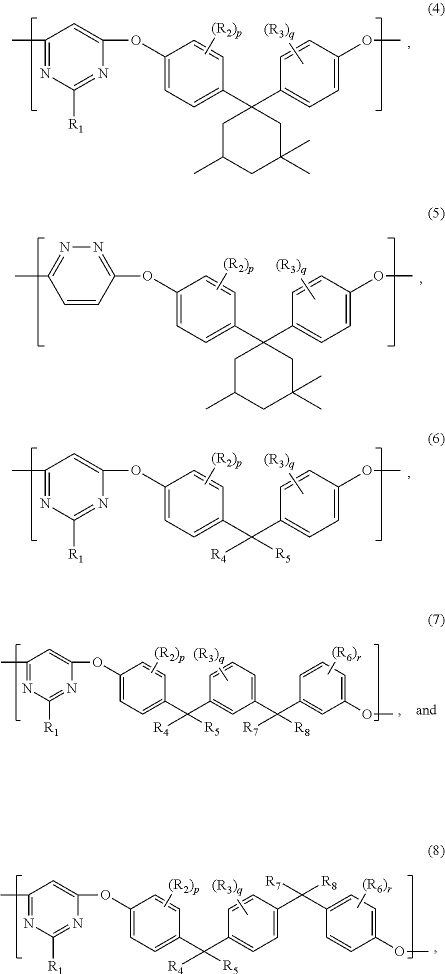
in which R₁ is H, C₁-C₁₀ alkyl, or aryl; each of R₂, R₃, and R₆, independently, is C₁-C₁₀ alkyl; each of R₄, R₅, R₇, and R₈ is H or C₁-C₁₀ alkyl; p is an integer from 0 to 4; q is an integer from 0 to 4; and r is an integer from 0 to 4.

62. The composition of claim 55, wherein the at least one low dielectric loss polymer comprises a poly(phenylene ether) of formula (V): 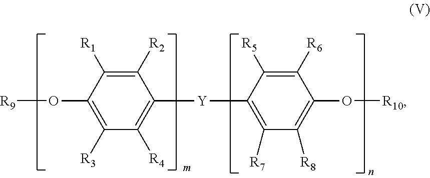
in which
each of m and n, independently, is an integer from 1 to 100;
each of R₁, R₂, R₃, R₄, R₅, R₆, R₇, and R₈, independently, is hydrogen or C₁-C₁₂ alkyl;
each of R₉ and R₁₀, independently, is an end group containing a carbon-carbon double bond; and
Y is a single bond, —C(O)—, —C(S)—, —S(O)—, —S(O)₂—, —C(RR′)—, or 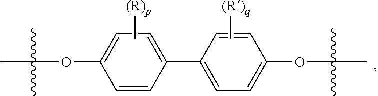
in which each of R and R′, independently, is hydrogen or C₁-C₁₂ alkyl, p is an integer from 0 to 4, and q is an integer from 0 to 4.

63. The composition of claim 55, wherein the at least one low dielectric loss polymer comprises a polyethylene, a polypropylene, a polybutadiene, a polystyrene, a poly(styrene-co-butadiene) copolymer, a polydivinylbenzene copolymer, a poly(styrene-ethylene-butylene-styrene) copolymer, a poly(styrene-ethylene-propylene-styrene) copolymer, a poly(styrene-ethylene-(ethylene-propylene)-styrene) copolymer, a poly(styrene-butadiene-styrene) copolymer, a poly(butadiene-styrene-butadiene) copolymer, a poly(styrene-isoprene-styrene) copolymer, a poly(styrene-propylene-styrene) copolymer, and a poly(ethylene-propylene-diene) copolymer.

64. The composition of claim 55, wherein the at least one low dielectric loss polymer is present in an amount of from about 1 wt % to about 30 wt % of the solid content of the composition.

65. The composition of claim 55, wherein the at least one filler comprises silica, alumina, quartz, titanium dioxide, boron nitride, barium titanate, barium strontium titanate, or polymer particles.

66. The composition of claim 55, wherein the at least one filler comprises hollow particles.

67. The composition of claim 66, wherein the hollow particles comprise hollow silica particles or hollow polymer particles.

68. The composition of claim 55, wherein the particles have an average diameter from about 0.5 μm to about 10 μm.

69. The composition of claim 55, wherein the at least one filler is present in an amount of from about 10 wt % to about 85 wt % of the solid content of the composition.

70. The composition of claim 55, wherein the at least one radical initiator comprises a peroxide, a hydrocarbon, or an azo compound.

71. The composition of claim 70, wherein the at least one radical initiator comprises 2,2′-azobis(2,4,4-trimethylpentane), di-(tert-butylperoxyisopropyl)benzene, bis(1-methyl-1-phenylethyl) peroxide, 2,3-dimethyl 2,3-diphenyl butane, or dicumyl peroxide.

72. The composition of claim 55, wherein the at least one radical initiator is present in an amount of from about 0.1 wt % to about 10 wt % of the solid content of the composition.

73. The composition of claim 55, further comprising at least one organic solvent.

74. The composition of claim 73, wherein the at least one organic solvent comprises tetrahydrofuran, acetonitrile, dimethylformamide, N-methylpyrrolidone, dimethylacetamide, acetone, 2-heptanone, methyl ethyl ketone, methyl isobutyl ketone, methyl n-amyl ketone, methyl isoamyl ketone, cyclopentanone, cyclohexanone, benzene, anisole, toluene, 1,3,5-trimethylbenzene, xylene, propylene glycol monomethyl ether, propylene glycol monomethyl ether acetate, or a combination thereof.

75. The composition of claim 73, wherein the at least one organic solvent is present in an amount of from about 1 wt % to about 50 wt % of the total weight of the composition.

76. The composition of claim 55, further comprising at least one coupling agent.

77. The composition of claim 76, wherein the at least one coupling agent comprises a silane, a siloxane, a polymethylsilsesquioxane, a titanate, or a zirconate.

78. The composition of claim 77, wherein the at least one coupling agent comprises diethoxymethylvinylsilane, trimethoxy(7-octen-1-yl)silane, allyltrimethoxysilane, methacryloxypropyltrimethoxysilane, vinyltrimethoxysilane, triethoxyvinylsilane, hydrolyzed vinylbenzylaminoethylaminopropyltrimethoxy silane, phenyltrimethoxysilane, (p-methylphenyl)trimethoxysilane, p-styryltrimethoxysilane, 3-isocyanatepropyltriethoxysilane, 3-methacryloxypropyl trimethoxysilane, amino ethyl amino trimethoxy silane, amino ethyl amino propyl trimethoxy silane tridecafluoro-1,1,2,2-tetrahydrooctyl(triethoxy), tetra(2,2-diallyloxymethyl-1-butyl)bis(ditridecylphosphite)-titanate, or tetra(2,2-diallyloxymethyl-1-butyl)bis(ditridecylphosphite)zirconate.

79. The composition of claim 76, wherein the at least one coupling agent is present in an amount of from about 0.1 wt % to about 10 wt % of the solid content of the composition.

80. The composition of claim 55, further comprising a flame retardant.

81. The composition of claim 80, wherein the flame retardant comprises 1,1′-(ethane-1,2-diyl)bis(pentabromobenzene), N,N-ethylene-bis(tetrabromophthalimide), aluminum diethylphosphinate, p-xylylene-bis-diphenylphosphine oxide, (2,5-diallyloxyphenyl)diphenylphosphine oxide, hexaphenoxy cyclotriphosphazene, tris(2-allylphenoxy)triphenoxy cyclotriphosphazene, resorcinol bis(di-2,6-dimethylphenyl phosphate), an allyl-containing phosphazene, or a vinyl containing phosphazene.

82. The composition of claim 80, wherein the composition comprises first and second flame retardants, the first flame retardant is insoluble in an organic solvent, and the second flame retardant is soluble in an organic solvent.

83. The composition of claim 82, wherein the weight ratio of the first flame retardant to the second flame retardant is from about 0.5:1 to about 3:1.

84. The composition of claim 80, wherein the flame retardant is present in an amount of from about 1 wt % to about 45 wt % of the solid content of the composition.

85. The composition of claim 55, further comprising at least one cross-linking agent.

86. The composition of claim 85, wherein the at least one cross-linking agent comprises 1,2-bis(4-vinylphenyl)ethane, triallyl cyanurate, triallyl isocyanurate, trimethylallyl isocyanurate, an alkyl functionalized allylsubstituted triazine, a polybutadiene, a poly(butadiene-co-styrene) copolymer, divinylbenzene, a styrene-divinylbenzene copolymer, an ethylene-styrene-divinylbenzene terpolymer, a di(meth)acrylate, bisphenol A diallyl ether, acenapthylene, a cyanate ester, a benzoxazine, or a bismaleimide.

87. The composition of claim 85, wherein the at least one cross-linking agent is present in an amount of from about 1 wt % to about 20 wt % of the solid content of the composition.

88. A film prepared from the composition of claim 55.

89. The film of claim 88, wherein the film is a free standing film.

90. The film of claim 88, wherein the film has a tensile modulus of at most about 10000 MPa.

91. The film of claim 88, wherein the film has a dissipation factor of at most about 0.004.

92. An article, comprising:
a carrier; and
at least one layer supported by the carrier, the at least one layer comprising the film of claim 88.

93. The article of claim 92, wherein the carrier comprises a metal foil, paper, or a polymer.

94. The article of claim 93, wherein the metal foil is a stainless steel foil, a copper foil, an aluminum foil, or an aluminum foil treated with palladium.

95. A laminate, comprising:
first and second films or a plurality of films; and
a woven or non-woven substrate between the first and second films;
wherein at least one of the first and second films is the film of claim 88.

96. The laminate of claim 95, further comprising a metal foil on a surface of the first or second film.

97. The laminate of claim 96, further comprising first and second metal foils;
wherein the first film is between the first metal foil and the substrate and the second film is between the second metal foil and the substrate.

98. The laminate of claim 97, wherein the substrate comprises a glass cloth.

99. A circuit board for use in an electronic product, comprising the laminate of claim 97.

100. A curable composition, comprising:
at least one maleimide-containing compound comprising a bismaleimide compound or a polymaleimide;
at least one filler;
at least one radical initiator; and
at least one organic solvent.
